(19) United States
(12) Patent Application Publication
(10) Pub. No.: US 2021/0369738 A1
(43) Pub. Date: Dec. 2, 2021

(54) PRODUCTION METHOD OF TELOMERASE ACTIVATORS AND TELOMERASE ACTIVATORS OBTAINED BY THIS METHOD

(71) Applicants: IZMIR YUKSEK TEKNOLOJI ENSTITUSU, Izmir (TR); EGE UNIVERSITESI REKTORLUGU, Izmir (TR)

(72) Inventors: Erdal BEDIR, Izmir (TR); Petek BALLAR KIRMIZIBAYRAK, Izmir (TR); Guner EKIZ, Izmir (TR); Sinem YILMAZ, Izmir (TR); Seda DUMAN, Izmir (TR); Melis KUCUKSOLAK, Izmir (TR)

(73) Assignees: IZMIR YUKSEK TEKNOLOJI ENSTITUSU, Izmir (TR); EGE UNIVERSITESI REKTORLUGU, Izmir (TR)

(21) Appl. No.: 16/753,346

(22) PCT Filed: Oct. 2, 2018
(86) PCT No.: PCT/TR2018/050540

§ 371 Date: Apr. 3, 2020

(30) Foreign Application Priority Data
Oct. 4, 2017 (TR) 2017/14942

(51) Int. Cl. A61K 31/58; A61K 36/481; C12N 1/14

(52) U.S. Cl. CPC A61K 31/58; C12N 1/14; A61K 36/481

(57) ABSTRACT

A method for producing telomerase activators which provide to obtain new/novel molecules (metabolites) from saponin group compounds by using biotransformation with endophytic fungi and telomerase activators obtained by this method. Included is the elucidation of chemical structures and investigation of the effects of telomerase enzyme activation in cells. These molecules have the potential to be used in diseases and/or conditions that can be treated/ameliorated by telomerase activation and associated with telomere shortening (For example; HIV, degenerative diseases, acute and chronic wound healing, ex vivo cell therapies and stem cell proliferation due to increment in vitro and ex-vivo replicative capacity of cells).

### CROSS REFERENCES TO THE RELATED APPLICATIONS

This application is the national phase of International Application No. PCT/TR2018/050540, filed on Oct. 2, 2018, which is based upon and claims priority to Turkish Patent Application No. 2017/14942, filed on Oct. 4, 2017, the entire contents of which are incorporated herein by reference.

### TECHNICAL FIELD

The present invention relates to a method for producing telomerase activators which provide to obtain new/novel molecules (metabolites) from saponin group compounds by using biotransformation with endophytic fungi and telomerase activators obtained by this method.

### BACKGROUND

Saponins are secondary metabolites carrying sugar units on a triterpenic or steroidal core with a high molecular weight and broad distribution in the plant kingdom. Saponin rich plants have been known for centuries as the main components of traditional medicine and are used in the treatment of various diseases. Cycloartane-type triterpenoids (9β, 19-cyclolanostane), produced only by photosynthetic eukaryotes, were first described in Astragalus plants. This group of compounds has made the Astragalus species a focus of interest, in terms of its richness and in particular, the determination of bioactivity at the molecular level. The biological activities of cycloartanes and their semi-synthetic derivatives are particularly showed cholesterol lowering, anticarcinogenic and immunomodulatory effects [1] [2].

The most important development that led to gain high commercial value of Astragalus cycloartanes was discovery and development of cycloastragenol, aglycone of many Astragalus cycloartane glycosides, as a telomerase activator (U.S. Pat. No. 7,846,904 B2). Geron Corp. (2004) discovered the presence of this compound (Cycloastragenol=CA) in Astragalus membranaceus, and cycloastragenol was licensed by TA Sciences in 2007 and supplied to the dietary supplement market as a new anti-aging product with the trade name TA-65 [3].

TA-65 is currently the only natural product as a telomerase activator in the market. A randomized double-blind, placebo-controlled clinical trial has shown that TA-65 extends human telomere and contributes to healthy aging without any product-related toxicity [4].

A recent study showed that TA-65 is also effective in the treatment of macular degeneration with a randomized, placebo-controlled clinical trial. There are also patents relating to the cosmetic use of the compounds, including cyclostagenol (CA) and astragenol (AG) (US20070154435 A1; WO2005000248 A2, U.S. Pat. No. 9,248,088 B2).

Cycloastragenol has a low bioavailability (5%) as a telomerase activator. In both oral and topical applications, bioavailability enhancing formulations need to be developed. Also, when assessed for potency, it is not a very effective compound and high doses are needed for the effect. For these reasons, molecules with better water solubility, higher bioavailability and higher bioactivity at lower doses are needed. Further, in another study conducted to discover/develop new potent telomerase activators in response to the limited bioavailability of these natural compounds when taken orally in mammals; it has been found that formulations prepared for pharmaceutical applications of synthetic triterpenoids derived from these compounds enhance telomerase activity in cells (U.S. Pat. No. 8,481,721 B2). A clinical trial has been commenced on the use of a semi-synthetic cycloastragenol derivative in the metabolic syndrome.

Telomerase activators that can be used in mammalian species, orally or topically, with longer bioavailability and longer half-life, are needed as defined in the studies.

### SUMMARY

The object of the invention is to carry out the method of producing telomerase activators which have potential to be used in diseases and/or conditions (HIV, degenerative diseases, acute and chronic wound healing, ex vivo cell therapies due to increment of the replicative capacity of the cells in vitro and ex vivo and stem cell proliferation) that can be prevented/treated by telomerase activation, by carrying out microbial biotransformation of cycloartane-type sapogenols with endophytic fungi isolated from Astragalus plant.

Another object of the invention is to carry out a production method which provides the formation of telomerase activators which can be used orally or topically in mammalian species, with longer bioavailability and longer half-life.

### DETAILED DESCRIPTION OF THE EMBODIMENTS

“Production Method of Telomerase Activators and Telomerase Activators Obtained by This Method” to achieve the purpose of the present invention is shown in the attached figures, from these figures:

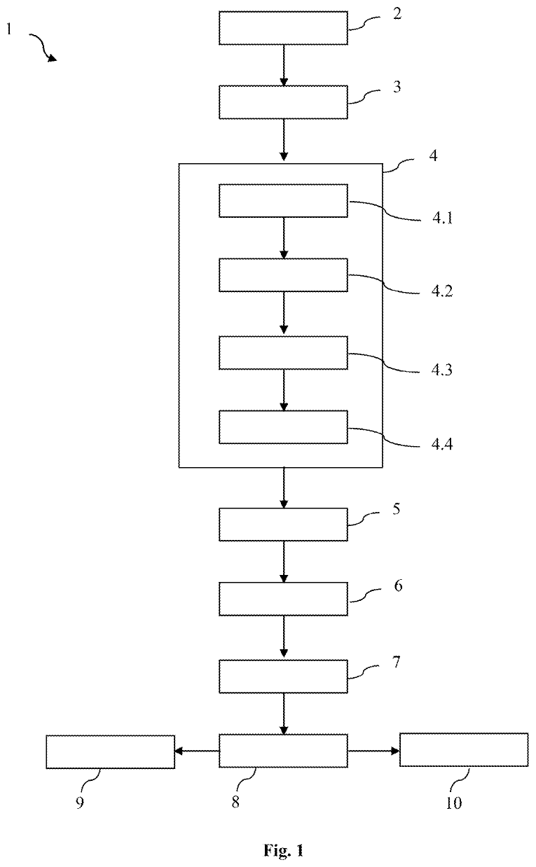

FIG. 1. It is a schematic representation of the isolation and purification of endophytic fungi from host plants.

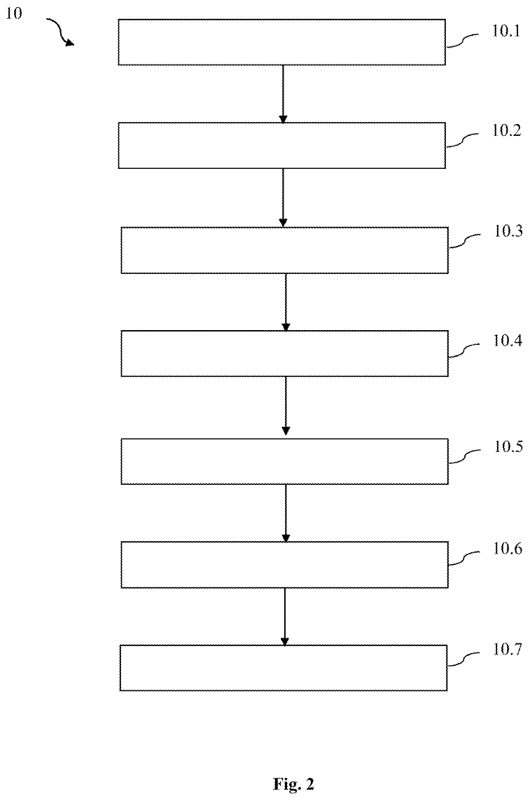

FIG. 2. It is a schematic representation of biotransformation screening studies.

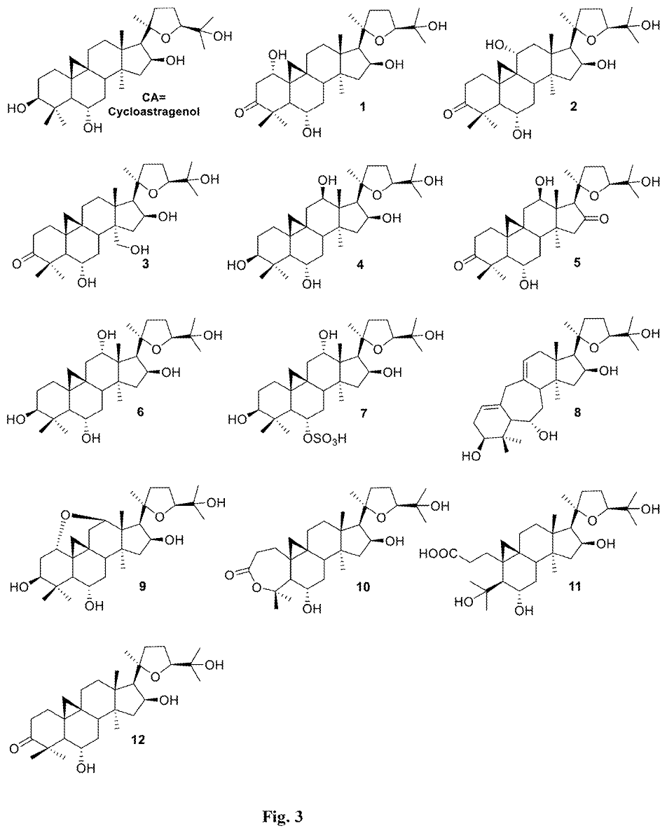

FIG. 3. It is an indication of formulas of CA and biotransformation products of CA.

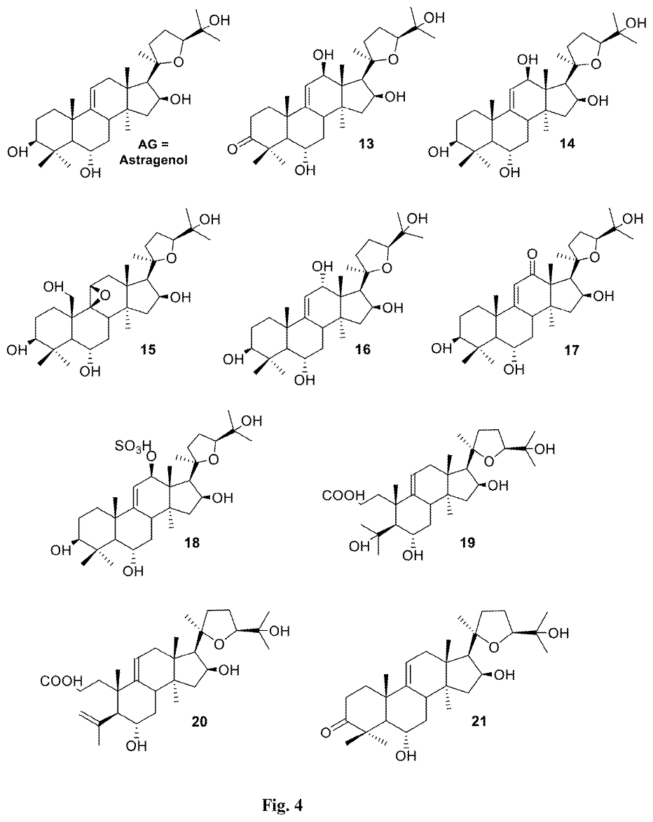

FIG. 4. It is an indication of formulas of AG and biotransformation products of AG.

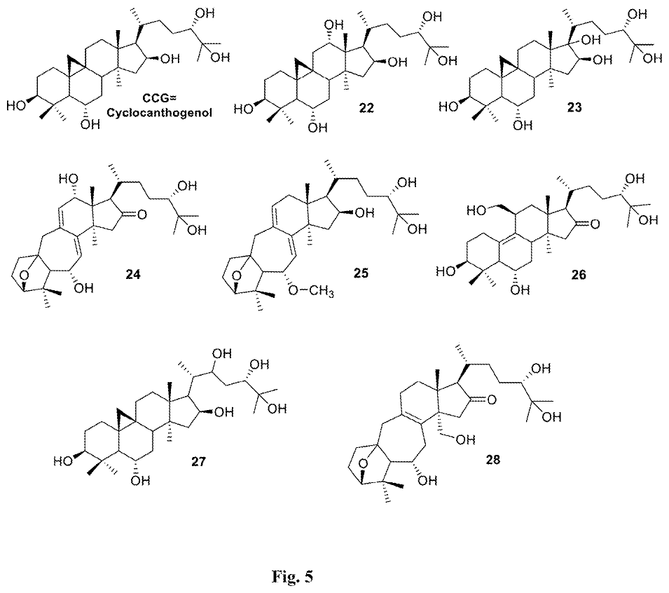

FIG. 5. It is an indication of formulas of CCG and biotransformation products of CCG.

The parts in the figures are numbered and their correspondence is given below.
1. Isolation and purification of endophytic fungi from host plants,
2. Preparation of plant materials,
3. Cutting of plant materials in appropriate dimensions (1-3 cm length) for washing and surface
sterilization,
4. Surface sterilization,
   4.1. Plant tissues are kept in 70% ethanol (5 minutes)
   4.2. Plant tissues are kept in 3-5% NaOCl (5 minutes)
   4.3. Plant tissues are kept in 70% ethanol (30 seconds)
   4.4. Storage of plant tissues in sterile dH₂O (3×1 min)
5. Drying of the samples in a laminar flow cabinet followed by removal of outer shells to cut fragments of internal tissues (approximately 0.5 cm²),
6. Inoculation into petri dishes containing PDA, MEA, RBC agar and WA,
7. 4-6 weeks incubation at 25° C.,
8. Ensuring the continuity of cultures by inoculating the axenic cultures into petri dishes containing fresh media,
9. Performing identification studies,
10. Carrying out biotransformation studies,
   10.1. Preparation of inoculum (PDA medium, 25° C., 5 days)
   10.2. Inoculation to liquid medium (Biotransformation media/PDB, 50 ml liquid media, 25° C., 180 rpm, 21 days)
   10.3. Addition of 10 mg substrate to the medium after 72 hours of inoculation
   10.4. Centrifuge for 10 minutes at 10000 rpm with 1 ml of sample taken on days 0, 2, 4, 6, 8, 10, 12, 14, 17 and 21
   10.5. Extraction of the supernatant with 1:1 EtOAc.
   10.6. Controlling formation of the new metabolites by comparing the samples with reference compounds on the TLC
   10.7. Transfer of production protocols affording the new metabolite formation to the preparative scale.

The present invention includes the method of production of telomerase activators, provides for the production of new/novel molecules that can be used in diseases and/or conditions which can be treated/ameliorated by telomerase activation by carrying out microbial biotransformation on saponins from triterpenes group with endophytic fungi, consists of the following steps:
washing plant material under tap water, cutting in appropriate sizes for surface sterilization (3-5 cm length) and washing with distilled water,
keeping the washed plant tissues in 70% ethanol for 5 min followed by 5 min in 3-5% sodium hypochlorite (NaOCl) (5% for root, 3% for stem, leaves and flowers),
keeping plant tissues in 70% ethanol for 30 seconds and waiting for 1 minute in three separate containers containing sterile distilled water,
drying of the tissues in the laminar flow chamber and removal of the outer shells under aseptic conditions,
cutting of internal tissues into small pieces (about 0.5 cm²) and placing in petri dishes containing four different media [PDA (Potato Dextrose Agar), MEA (Malt Extract Agar), RBC (Rose Bengal Chloramphenicol) agar and WA (Water Agar)] and ensuring incubation for 4-6 weeks at 25° C.,
transfer of 100 □l of the water used in the final wash step of the plant material into PDA, MEA, RBC agar and WA media, spreading with a glass baguette to the medium on the petri dish, incubation with other petri dishes,
transfer of fungal hyphae (fungal structure/filament) observed to develop from the plant tissues during the incubation phase to fresh petri dishes for the purification,
transfer of fungal hyphae to fresh media, coding of the axenic cultures (pure cultures/fungal isolates which are ensured to be pure by repeated sub culturing) based on the isolated host plant species (1E3B, 1E4A and 1E4C codes are used for Astragalus condensatus; 1E1A, 1E1B, 1E2, 1E2A, 1E3A and 1E4B codes are used for Astragalus angustifolius) and plant tissue (R: root, S: stem, L: leaf), routinely ensuring the continuity of cultures by inoculating in petri dishes containing PDA (Potato Dextrose Agar) medium,
autoclaving of 4 ml PDA media with glass tubes (with cap, 16×100 mm) to keep isolates at +4° C., preparation of stock cultures by inoculating to mediums which are solidified as a flat agar,
incubation of fungal isolates in petri dishes containing YM 6.3 (Yeast-Malt Medium, pH 6.3) medium for 15 days at 23° C. for the identification studies,
incubating fungal isolates in petri dishes containing PDA for 5 days at 25° C. for biotransformation studies,
inoculation of the incubated fungi into a biotransformation medium as a suspension culture,
after inoculation, dissolving the substrate [CA (Cycloastragenol), AG (astragenol) or CCG (Cycloanthogenol)] in DMSO (Dimethyl sulfoxide) and adding to the biotransformation medium and maintaining the incubation in submerged culture conditions,
biotransformation of substrate (CA, AG, CCG) with fungal isolate in biotransformation medium,
removal of cells from the production broth under vacuum after incubation, followed by extraction of the resulting filtrate with an equal volume of EtOAc,
combining the EtOAc phases and treating with anhydrous Na₂SO₄, then evaporation on a rotary evaporator at 40° C.,
obtaining telomerase activators that are new/novel molecules as final product.

In one embodiment of the invention, a medium containing a rich/low nutrient, containing antibiotics and containing PDA (Potato Dextrose Agar), MEA (Malt Extract Agar), RBC (Rose Bengal Chloramphenicol) agar and WA (Water Agar) is used in the fungus isolation in order to increase the endophytic isolation efficiency in the step of “cutting of internal tissues into small pieces, placing in petri dishes containing nutrient medium and ensuring incubation”.

In one embodiment of the invention, the biotransformation medium is a broth containing 2% D (+) glucose, 0.5% yeast extract, 0.5% NaCl, 0.5% K₂HPO₄ (w/v) or Potato Dextrose Broth (PDB) in the step of “inoculation of the incubated fungi into a biotransformation medium as a suspension culture”.

In one embodiment of the invention, different tissues of Astragalus condensatus and Astragalus angustifolius such as root, stem, leaf and flower are used as plant materials. The formulas of the substrates (CA (Cycloastragenol), AG (astragenol), CCG (Cycloanthogenol)) obtained from these plants are given below:

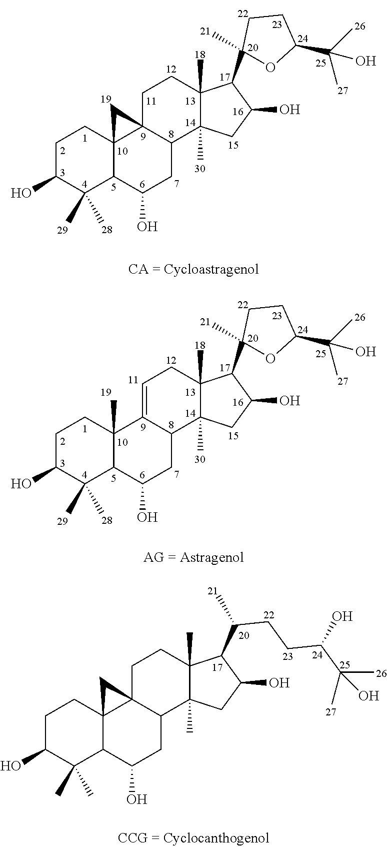

In one embodiment of the invention, the substrate [20% of the broth volume (w/v); 0.2 mg/ml; CA, AG and CCG] is dissolved in DMSO and added to the medium and biotransformation studies are carried out at 25° C. and 180 rpm at submerged culture conditions in the step of “after inoculation, dissolving the substrate in DMSO and adding to the biotransformation medium and maintaining the incubation in submerged culture conditions”.

In one embodiment of the invention, biotransformation of the substrate with the fungal isolate takes 10 days at 25° C. and a shaking speed of 180 rpm in a biotransformation medium [2% D (+) glucose, 0.5% yeast extract, 0.5% NaCl, 0.5% KJ-IPO′ (w/v)] in the step of “biotransformation of substrate with fungal isolate in biotransformation medium”.

In one embodiment of the invention, biotransformation of CA with at least one of the fungal isolates identified as Alternaria eureka, Neosartorya hiratsukae and Camarosporium laburnicola is performed in the step of “biotransformation of substrate with fungal isolate in biotransformation medium”.

In one embodiment of the invention, biotransformation of AG with at least one of the fungal isolates identified as Alternaria eureka and Camarosporium laburnicola is performed in the step of “biotransformation of substrate with fungal isolate in biotransformation medium”.

In one embodiment of the invention, biotransformation of CCG with the fungal isolate identified as Alternaria eureka is performed in the step of “biotransformation with fungal isolate with the substrate in the biotransformation medium”.

The telomerase activators obtained by the telomerase activator production method of the present invention are novel molecules which effectively increase telomerase activity when given to cells or tissues and the formulas of these new molecules (1, 3, 4, 6, 10, 11, 12, 14, 16, 17, 19, 20, 21) are given below:

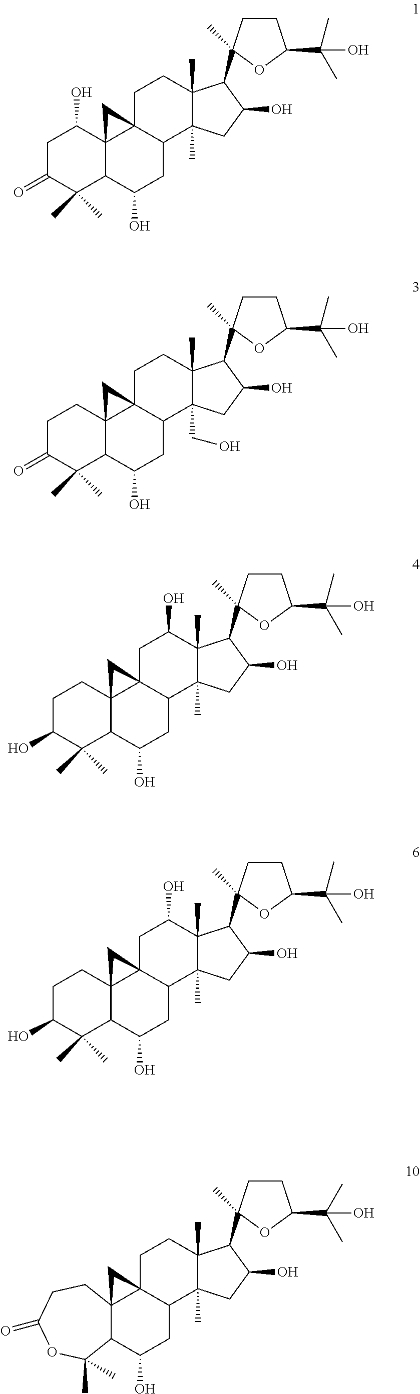

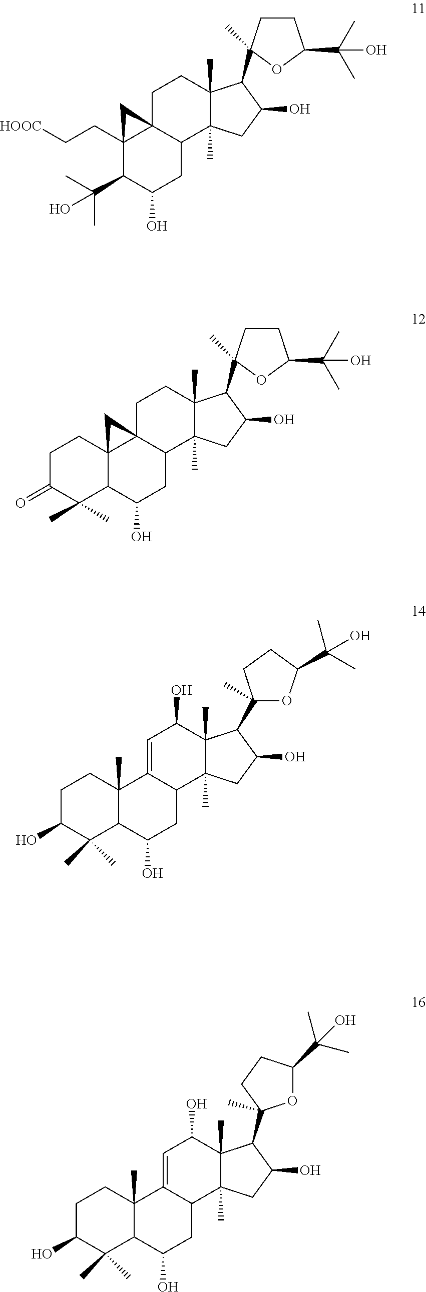

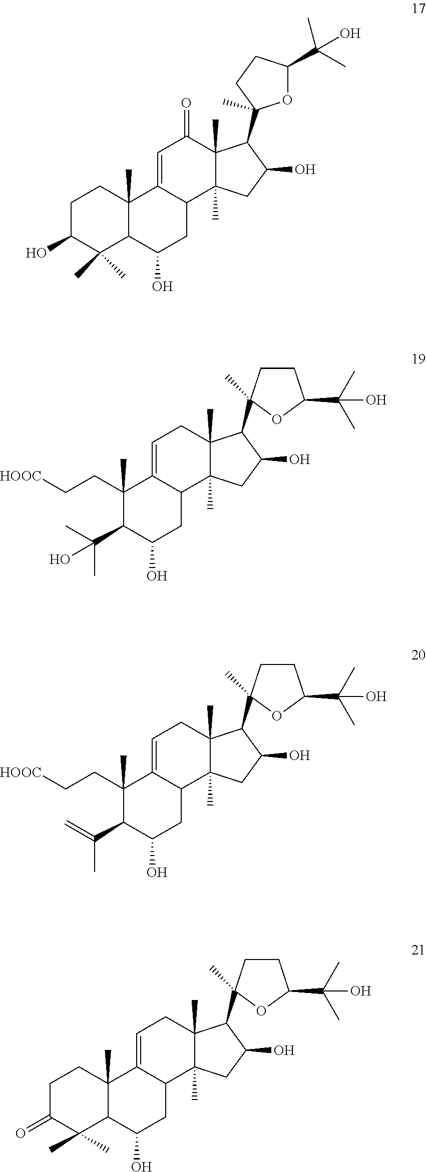

In one embodiment of the invention, the pharmaceutically acceptable salts of the molecules of the above formulas (1, 3, 4, 6, 10, 11, 12, 14, 16, 17, 19, 20, 21) are used as telomerase activators because they can effectively increase telomerase activity when given to cells or tissues.

Telomerase activators, which are novel molecules produced in the scope of the invention and their salts is used to prevent/treat a condition or disease in mammalian cells or tissues that require increased telomerase activation. These new molecules and their salts as telomerase activators are evaluated during in vitro production of stem cells or biological drugs (protein, antibody, etc.) used for regenerative or therapeutic purposes.

The telomerase activators obtained by the telomerase activator production method of the present invention are novel molecules which effectively increase telomerase activity when given to cells or tissues and the formulas of these new molecules (29-59) are given below:

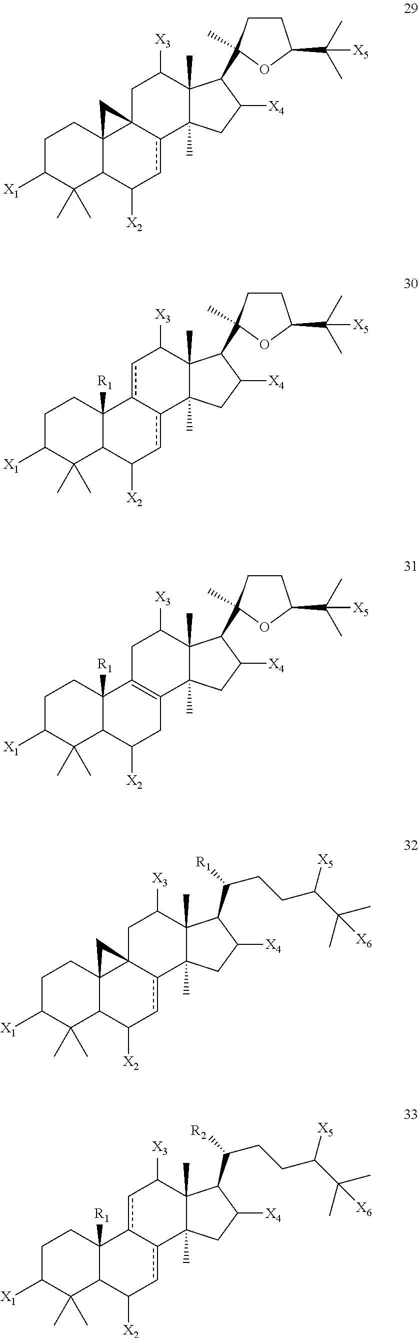

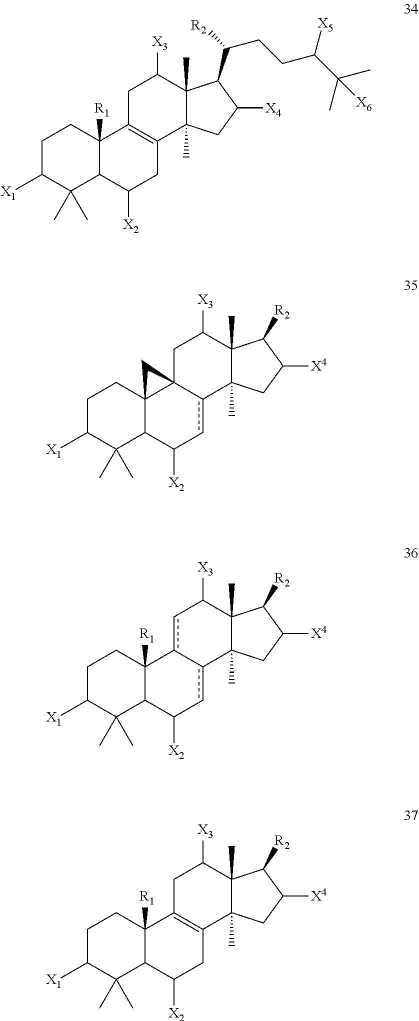

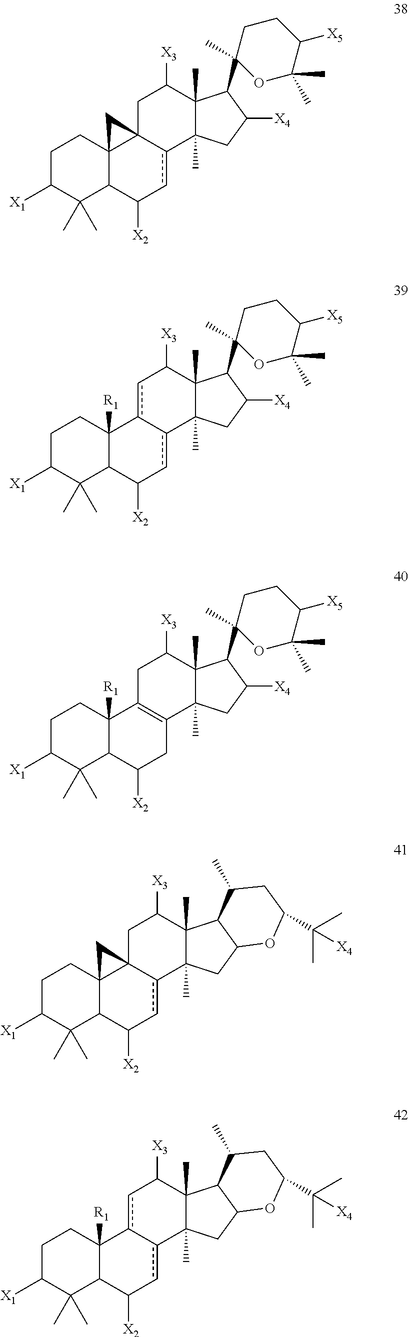

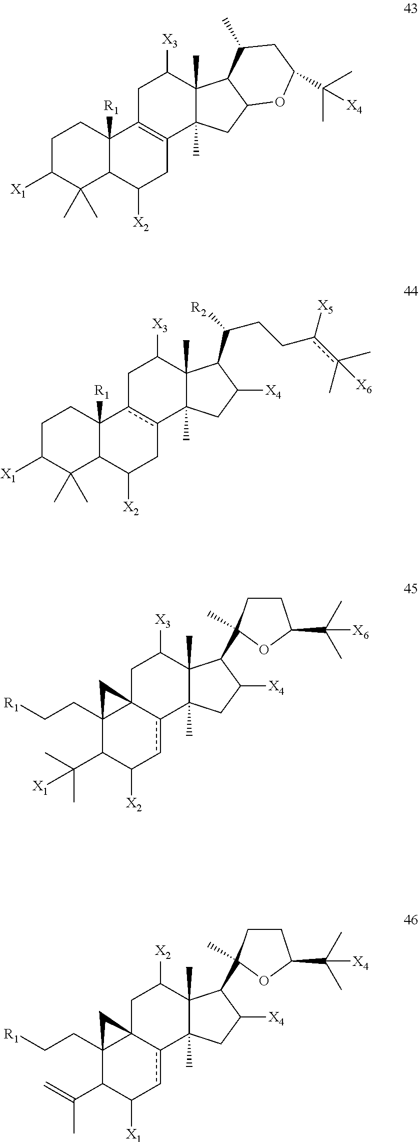

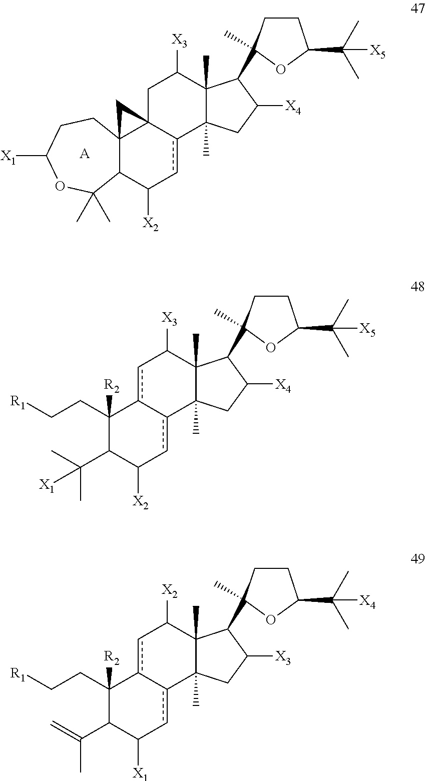

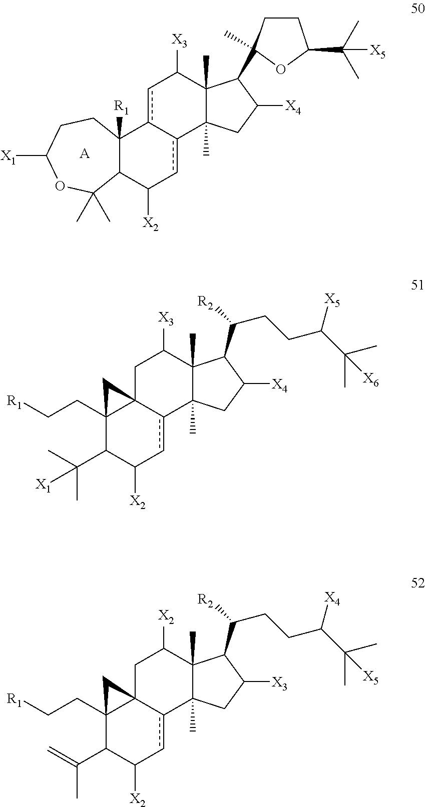

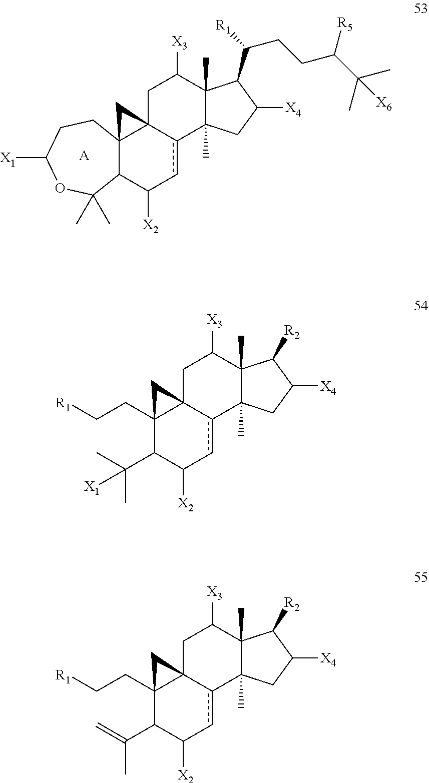

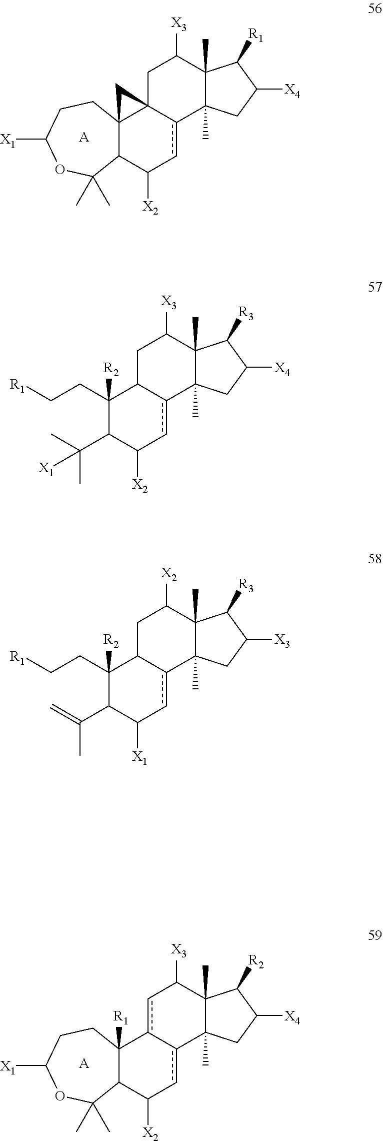

wherein each X₁, X₂, X₄, X₅ and X₆ are independently selected from hydrogen, hydroxy, alkoxy containing 1-6 carbons, acyloxy containing 1-6 carbons, keto and glycosides,
wherein X₃ is independently selected from hydroxy, alkoxy containing 1-6 carbons, acyloxy containing 1-6 carbons, keto and glycoside,
wherein each X₁, X₂, X₃, X₄, X₅ and X₆ independently have alpha and beta configuration,
if glycosylation is present on hydroxy groups, there may be new glycosylation on the sugar unit which directly attached main skeleton and the number of sugars on the glycosidic chain can extend to a total of 3,
wherein the groups R₁, R₂ and R₃ are independently selected from methyl and alcohol, aldehyde and carboxylic acid derivatives of this methyl having different oxidation level forms,
wherein R₁, R₂ and R₃ are independently selected from alkoxy containing 1-6 carbons, acyloxy containing 1-6 carbons and glycoside on the hydroxy group present if the groups R₁, R₂ and R₃ are in the form of primary alcohols,
wherein R₁, R₂ and R₃ are independently selected from ester or amide form with 1-16 carbon-bearing alcohols or amines if the groups R₁, R₂ and R₃ are in the form of carboxylic acid,

If glycosidation is present on the primary alcohol present on the R₁, R₂ and R₃ groups, there may be new glycosylation on the sugar unit which directly attached main skeleton and the number of sugars on the glycosidic chain can extend to a total of 3,
wherein the R₁ group in structure 35 and the R₂ group in structures 36-37 are independently selected from hydrogen, hydroxy, alkyl chain containing from 2 to 6 carbons, haloalkyl chain containing 2-6 carbons, aryl, heteroaryl, monocyclic cycloalkyl chain containing from 3 to 8 carbons, bicyclic cycloalkyl chain containing from 4 to 8 carbons, the heterocyclic ring in the monocyclic structure containing 3-8 carbons, the heterocyclic ring in the bicyclic structure containing 4-8 carbons and these chains may undergo substitution from 1, 2, 3 different points through the carbon atoms in the chain and this substitution may be an alkyl substitution containing from 1 to 3 carbons,
there may be a single or double bond between the ring carbons in the positions where the [symbol] symbol is present,
in the compounds 47, 50, 53, 56 and 59, the C-X₁ linkage extending from ring A can be a double bond and this double bond can occur with one of the oxygen, nitrogen or sulfur atoms, The telomerase activators, which are the novel molecules and their salts produced in the scope of the invention are used in the prevention or treatment of conditions or diseases present in a group of these diseases selected from the following or combinations thereof: viral infections, opportunistic infections, HIV, degenerative diseases, neurodegenerative diseases, degenerative diseases in bone, connective tissues and joints, diabetic retinopathy, macular degeneration, cardiovascular diseases, central and peripheral vascular diseases, Crohn's disease, immunological conditions, liver diseases, fibrosis, cirrhosis, lung diseases, pulmonary fibrosis, asthma, emphysema, chronic obstructive pulmonary diseases, hematopoietic disorders, anemia, thrombocytopenia, neutropenia, cytopenia, chronic inflammatory gastrointestinal diseases, Barret's esophagus, conditions associated with reduced proliferative capacity in stem cell or progenitor cells, bone marrow suppression diseases, aplastic anemia, myelodysplastic anemia, myelodysplastic syndrome, wounds, mucosal ulceration, keloid formation, hair loss, pigment loss, deep erosions and lesions, as well as severe acute and chronic discomforts, burns, abrasions, clefts and cuts, grafts, lesions, chronic venous ulcers, diabetic ulcers, cancer, genomic instability or increased mutations associated with telomerase or shortened telomer in pre-cancer cases, loss of tumor suppressor functions.

The subject of this invention is to obtain new/novel molecules from the compounds of saponin group by biotransformation with endophytic fungi, elucidate chemical structures of these compounds and increase telomerase enzyme activation in cells. These molecules have the potential to be used in diseases and/or conditions that can be treated/ameliorated by telomerase activation and associated with telomerase shortening (For example; HIV, degenerative diseases, acute and chronic wound healing, ex vivo cell therapies and stem cell proliferation due to increment in vitro and ex-vivo replicative capacity of cells).

The subject invention is based on microbial biotransformation, another method which can be used as an alternative to semi-synthesis to form a compound pool of high chemical diversity for bioactivity screening. In the method of the invention, it is intended to obtain new/novel derivatives from the compounds of the group of triterpenic saponins. In the scope of the invention, studies carried out on the saponins of the cycloartane group (cycloastragenol, cyclocanthogenol and astragenol) used as the starting molecule are not available in the literature. The use of endophytic fungi living inside the Astragalus species and Astragalus originated starting molecules has never been applied to make such modifications.

In the scope of the invention, microbial biotransformation studies are carried out by endophytic fungi isolated from Astragalus plant on cycloartane-type sapogenols in the group of triterpenic saponins and metabolites (new/novel molecules) which can significantly increase the activation of telomerase enzyme are obtained. Metabolites obtained by microbial biotransformation of cycloastragenol (CA) and astragenol (AG) as a starting compound with fungal endophytes were found to increase the activity of telomerase enzyme in HEKn cell line (Human Primer Epidermal Keratinocyte, ATCC; PCS-200-010) up to 11.3-fold. These molecules have the potential to be used in diseases and/or conditions that can be treated/ameliorated by telomerase activation and associated with telomerase shortening (For example; HIV, degenerative diseases, acute and chronic wound healing, ex vivo cell therapies and stem cell proliferation due to increment in vitro and ex-vivo replicative capacity of cells).

The invention relates to a method of producing a telomerase activator, biotransformation with endophytic fungi to obtain new/novel molecules from the saponins from natural sources, elucidation of their chemical structures, and methods for discovery of molecules that increase telomerase enzyme activation. Endophytic fungi, Alternaria eureka, Neosartoria hiratsukae and Camarosporium laburnicola which are used for biotransformation of the starting molecules (CA, AG, CCG) are isolated from the different tissues of plants of Astragalus condensatus and Astragalus angustifolius.

Telomerase activators that can be used in mammalian species, orally or topically, with longer bioavailability and longer half-life are needed. Microbial biotransformation studies with endophytic fungi isolated from Astragalus plant were carried out on the cycloartane type sapogenols in the group of triterpenic saponin and 28 metabolites, 18 of which were new, were obtained. The obtained metabolites were screened for the activation of telomerase enzyme. Some of the metabolites obtained by microbial biotransformation of our starting compounds, cycloastragenol (CA) and astragenol (AG), with fungal endophytes were found to increase the activity of telomerase enzyme in HEKn cell line (Human Primer Epidermal Keratinocyte, ATCC; PCS-200-010) at low doses up to 11.3-fold. These molecules have the potential to be used in diseases and/or conditions that can be treated/ameliorated by telomerase activation and associated with telomerase shortening (For example; HIV, degenerative diseases, acute and chronic wound healing, ex vivo cell therapies and stem cell proliferation due to increment in vitro and ex-vivo replicative capacity of cells).

When “effective to increase telomerase activity in a cell” is used for a compound, it is expressed that the compound increases telomerase activity in a keratinocyte or fibroblast cell line at a concentration of 1 μM or less at least 1.5-fold relative to the control. Herein the control is the level of telomerase activation produced by a similar formulation without the compound. In this study, cells treated with DMSO (dimethyl sulfoxide) were taken as controls. These molecules are of particular interest to pharmaceutical, biological drug production and cosmetic industry as they have the potential to be used in diseases and/or conditions that can be treated/ameliorated by telomerase activation and associated with telomerase shortening.

Experimental Work

Plant materials brought to the laboratory and washed under tap water are cut in appropriate sizes for surface sterilization and washed with distilled water. The washed plant tissues are kept in 70% ethanol for 5 minutes and then kept for 5 minutes in 3-5% sodium hypochlorite (NaOCl) (5% for root, 3% for branches, leaves and flowers). Plant tissues in 70% ethanol are kept for 30 seconds and then kept in three separate containers containing sterile distilled water for 1 minute. The tissues are dried in the sterilized cabin and after drying, the outer shells are removed with cutting tools such as bisturi under aseptic conditions. The inner tissues are divided into small pieces of about 0.5 cm² and placed in petri dishes with different media therein (PDA, MEA, RBC agar, WA). Plant tissues are allowed to incubate in petri dishes for 4 to 6 weeks at 25° C. For the control of surface sterilization, 100 μl of the water used for the final wash of the plant material is taken and then transferred to PDA, MEA, RBC agar and WA media and spread to the petri dish with glass baguette. Subsequently, incubation with other petri dishes is allowed.

The fungal hyphae observed during the incubation phase are transferred to petri dishes containing fresh medium for purification studies. In consideration of any possible contamination, purification is carried out in the petri dish as three replications. Axenic cultures are coded on the basis of the host plant species and plant tissues and are routinely inoculated into petri dishes containing PDA medium to ensure the continuity of cultures. Isolates are stored at 4° C. in the PDA medium by preparing the stock cultures. In FIG. 1, isolation and purification of endophytic fungi from host plants is schematized.

Identification studies of fungal endophytes are made using molecular methods based on rDNA ITS (ITS1 and ITS4 regions) sequence analysis. Fungal isolates are incubated for 15 days at 23° C. in petri dishes containing YM 6.3 medium. Genomic DNA extraction is performed using gene extraction kit. Polymerase chain reaction (PCR) is performed using ITS4 and ITS1F primers to amplify the ITS gene region in isolated DNA samples (Table 1).

TABLE 1
Primers used in PCR.

| Locus | Primer | Sequence 5′→3′ | Reference |
|---|---|---|---|
| ITS | ITS1F | 5′-CTT-GGT-CAT-TTA- | Gardens |
| | | GAG-GAA-GTA-A-3′ | and Bruns, |
| | | | 1993 |
| | ITS4 | 5′-TCC-TCC-GCT-TAT- | White et |
| | | TGA-TAT-GC-3′ | al., 1990 |

Type-based identification of the Alternaria eureka fungal isolate is carried out by sequence analysis of LSU and TEF1 [5] gene regions as well as ITS rDNA. The samples containing the PCR products are purified using the EZ-10 Spin Column PCR Purification Kit, and the purified PCR products are used in the sequence analysis. The resulting DNA sequences are processed using the Geneious® programme (Version 7.1.5), and then the species with the closest sequence in the database are identified using NCBI's (National Centre for Biotechnology Information) BLASTn (Nucleotide Basic Local Alignment Search Tool) tool.

Selected fungal isolates for biotransformation studies are inoculated into petri dishes containing PDA and incubated for 5 days at 25° C. Following incubation, the fungi are inoculated into the biotransformation medium [2% D (+) glucose, 0.5% yeast extract, 0.5% NaCl, 0.5% K₂HPO₄ (w/v)] with the aid of cork-borer (8 mm diameter) or as a suspension culture in Potato Dextrose Broth (PDB) (Sigma Aldrich, P6685-250G) medium. After 72 hours of inoculation, the substrate (CA, AG, CCG and 20(27)-nor CA; up to 20% (w/v) of the broth volume) is dissolved in DMSO and added to the medium and incubated at 25° C. and 180 rpm shaking speed in submerged culture conditions.

Biotransformation studies are carried out on two scales: “screening scale” and “preparative scale”. In all studies, two Erlenmeyers containing only microorganisms (microorganism+media) and containing only substrate (substrate+media) are used for control purposes. Screening studies are carried out in 250 ml of Erlenmeyer flasks containing 50 ml of medium and after 72 hours of inoculation, 10 mg of substrate is dissolved in 500 μl of DMSO and added to the media. 1 ml samples are taken at 0, 2, 4, 6, 8, 10, 12, 14, 17 and 21 days. The cells are removed by centrifugation at 10,000 rpm for 10 minutes and then an equal volume of ethyl acetate (EtOAc) is added, the extraction of the samples is carried out in the separation funnel. Screening scale studies involving the comparison of extracts with reference materials by thin layer chromatography (TLC), checking for the formation of new metabolites, and scaling up for endophytes providing different metabolites to the preparative scale are schematized in FIG. 2.

Preparative scale studies are carried out in the direction of the data obtained from the screening scale based on the time period in which the metabolite diversity is detected at the maximum. Preparative scale studies are performed in 1000 ml Erlenmeyer flasks containing 300 ml of medium. After 72 hours of inoculation, 60 mg substrate is added to the medium.

The preparative biotransformation study of CA (1200 mg) with the isolate known as Alternaria eureka is continued for 10 days at 25° C. and 180 rpm in a biotransformation medium. After incubation, cells are removed from the production medium under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract (1.79 g) is fractionated by column chromatography with Sephadex LH-20 (75 g) (100% MeOH). The fractions obtained are combined according to TLC profiles and further fractionated on 100 g RP (C-18). Thus, the isolation of metabolites A-CA-01 (5 mg) [6], A-CA-02 (2.3 mg), A-CA-03 (13.2 mg) [7], A-CA-04 (5.5 mg), A-CA-05 (4.5 mg) and A-CA-07 (3 mg) are carried out (FIG. 3).

The preparative biotransformation study of CA (1750 mg) with the isolate known as Neosartorya hiratsukae is continued for 10 days at 25° C. and 180 rpm in a biotransformation medium. After incubation, cells are removed from the production broth under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract (2.07 g) is fractionated by column chromatography with Silica gel (180 g). Fractions collected after silica gel column chromatography are combined according to their TLC profiles to proceed to further fractionation work. As a result of the isolation studies, Nh-CA-01 (5.2 mg), Nh-CA-02 (3.5 mg) and Nh-CA-03 (6.2 mg) [7] molecules are obtained purely (FIG. 3).

The preparative biotransformation study of CA (500 mg) with the isolate known as Camarosporium laburnicola is continued for 4 days at 25° C. and 180 rpm in a PDB. After incubation, cells are removed from the production broth under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract is fractionated by column chromatography with RP (C18) (70 g). Fractions are combined according to the TLC profiles. This fractionation yields pure metabolites E-CG-01 (160 mg) [8], E-CG-02 (60 mg) [6] and E-CG-03 (14 mg) [6] (FIG. 3).

The preparative biotransformation study of AG (1140 mg) with the isolate known as Alternaria eureka is continued for 10 days at 25° C. and 180 rpm in a biotransformation medium. After incubation, cells are removed from the production broth under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract (2.3 g) is fractionated by column chromatography with Sephadex LH-20 (75 g) (100% MeOH). The fractions are combined according to the TLC profiles and applied to column prepared with 100 g RP (C-18). Further chromatography studies are performed on the collected fractions. This fractionation yields pure A-AG-01 (6.4 mg), A-AG-02 (5.2 mg) [8], A-AG-03 (3.3 mg), A-AG-05 (10 mg) [9], A-AG-06 (6.6 mg) and A-AG-07 (13.7 mg) molecules (FIG. 4).

The preparative biotransformation study of AG (800 mg) with the isolate known as Camarosporium laburnicola is continued for 10 days at 25° C. and 180 rpm in a PDB. After incubation, cells are removed from the production broth under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract is fractionated by column chromatography with RP (C18) (70 g). Fractions are combined according to the TLC profiles. This fractionation yields pure metabolites E-AG-01 (6.3 mg) [6], E-AG-02 (17 mg) [8] and E-AG-03 (9.2 mg) (FIG. 4).

The preparative biotransformation study of CCG (1080 mg) with the isolate known as Alternaria eureka is continued for 10 days at 25° C. and 180 rpm in a biotransformation medium. After incubation, cells are removed from the production broth under vacuum using a Buchner funnel, and the resulting filtrate is then extracted with an equal volume of EtOAc. The EtOAc phases are combined and treated with anhydrous Na₂SO₄ and subsequently evaporated at 40° C. on a rotary evaporator. The EtOAc extract is fractionated by column chromatography with Sephadex LH-20 (70 g) (100% MeOH). The fractions are combined according to the TLC profiles and applied to column prepared with 100 g RP (C-18). Further chromatography studies are performed on the collected fractions. The molecules A-SKG-01 (22 mg), A-SKG-03 (22.2 mg), A-SKG-05 (8 mg), A-SKG-06 (8 mg), A-SKG-09 (5.2 mg), A-SKG-10 (6.4 mg) and A-SKG-11 (20 mg) are obtained in pure form (FIG. 5).

Characterization of all molecules obtained is elucidated using one- and two-dimensional nuclear magnetic resonance (1D-, 2D-NMR) and high-resolution mass spectroscopy (HR-MS). Below, the spectral data of the newly characterized and known molecules and references (for known molecules) are presented:

1: HRTOFMS m/z 527.33752 ([M+Na]⁺) C₃₀H₄₈O₆Na=527.33486. ¹H-NMR (500 MHz, CDCl₃) δ 4.73 (1H, ddd, J=8.0, 7.8, 6.8 Hz, H-16), 3.75 (1H, t, J=7.1 Hz, H-24), 3.65 (1H, d, J=9.1 Hz, H-1), 3.56 (1H, m, H-6), 2.58 (1H, m, H-22a), 2.43 (1H, d, J=7.8 Hz, H-17), 2.32 (1H, m, H-12a), 2.25 (1H, dd, J=14.2, 9.5 Hz, H-2a), 2.07 (1H, dd, J=12.6, 8.0 Hz, H-15a), 2.00 (2H, m, H-11), 2.00 (2H, m, H-23), 1.99 (1H, d, J=10.0 Hz, H-5), 1.80 (1H, d, J=14.5, H-2b), 1.68 (1H, m, H-8), 1.61 (1H, m, H-12b), 1.61 (1H, m, H-22b), 1.51 (2H, m H-7), 1.43 (1H, dd, J=13.8, 6.8 Hz, H-15b), 1.38 (3H, s, H-28), 1.31 (3H, s, H-27), 1.25 (3H, s, H-18), 1.23 (3H, s, H-21), 1.23 (3H, s, H-29), 1.23 (3H, s, H-30), 1.15 (3H, s, H-26), 0.95 (1H, d, J=4.4 Hz, H-19a), 0.79 (1H, d, J=4.4 Hz, H-19b); ¹³C-NMR (125 MHz, CDCl₃) δ 215.7 (s, C-3), 86.9 (s, C-20), 81.3 (d, C-24), 73.7 (d, C-1), 73.3 (d, C-16), 71.9 (s, C-25), 69.7 (d, C-6), 59.1 (d, C-17), 54.4 (d, C-5), 50.8 (s, C-4), 49.2 (d, C-8), 47.5 (t, C-15), 45.4 (s, C-13), 44.8 (s, C-14), 44.6 (t, C-2), 39.2 (t, C-7), 34.5 (t, C-22), 34.0 (t, C-12), 33.6 (t, C-19), 30.3 (s, C-10), 27.9 (q, C-21), 27.7 (q, C-27), 27.1 (q, C-28), 26.6 (q, C-26), 25.8 (t, C-11), 25.8 (t, C-23), 23.1 (s, C-9), 21.8 (q, C-18), 21.3 (q, C-29), 19.6 (q, C-30).

2: HRTOFMS m/z 527.33762 ([M+Na]⁺) C₃₀H₄₈O₆Na=527.33486. ¹H-NMR (400 MHz, CD₃OD) δ 4.67 (1H, ddd, J=7.2, 7.9, 7.9 Hz, H-16), 4.02 (1H, dd, J=9.2, 3.0 Hz, H-11), 3.77 (1H, m, H-24), 3.55 (1H, m, H-6), 2.66 (1H, m, H-2a), 2.42 (1H, dd, J=13.6, 10.2 Hz, H-12a), 2.39 (1H, m, H-2b), 2.36 (1H, d, J=8.0, H-17), 2.04 (2H, m, H-23), 2.01 (1H, m, H-1a), 2.01 (1H, m, H-5), 1.98 (1H, m, H-15a), 1.97 (1H, m, H-8), 1.91 (1H, m, H-1b), 1.65 (1H, m, H-22a), 1.61 (1H, m, H-22b), 1.59 (1H, dd, J=14.4, 3.2 Hz, H-12b), 1.47 (1H, m, H-7a), 1.44 (1H, m, H-15b), 1.43 (1H, m, H-7b), 1.34 (3H, s, H-28), 1.28 (3H, s, H-27), 1.26 (3H, s, H-18), 1.24 (3H, s, H-21), 1.23 (1H, m, H-19a), 1.22 (3H, s, H-29), 1.13 (3H, s, H-26), 0.95 (3H, s, H-30), 0.41 (1H, d, J=4.4 Hz, H-19b); ¹³C-NMR (100 MHz, CD₃OD) δ 220.8 (s, C-3), 88.2 (s, C-20), 82.7 (d, C-24), 74.5 (d, C-16), 72.6 (s, C-25), 69.7 (d, C-6), 65.0 (d, C-11), 59.1 (d, C-17), 55.0 (d, C-5), 51.5 (s, C-4), 49.0 (d, C-8), 48.2 (t, C-12), 47.0 (s, C-13), 46.7 (t, C-15), 46.3 (s, C-14), 38.6 (t, C-7), 36.7 (t, C-2), 35.5 (t, C-22), 30.2 (t, C-1), 29.6 (s, C-10), 28.7 (s, C-9), 28.5 (q, C-21), 28.5 (q, C-28), 27.6 (q, C-27), 26.8 (t, C-23), 26.7 (q, C-26), 22.4 (t, C-19), 21.6 (q, C-30), 20.5 (q, C-18), 20.5 (q, C-29).

3: ¹H-NMR (400 MHz, pyridine-d₅) δ 5.27 (1H, m, H-16), 4.18 (1H, d, J=11.6 Hz, H-30a), 4.10 (1H, d, J=11.6 Hz, H-30b), 3.94 (1H, dd, J=5.2, 9.0 Hz, H-24), 3.72 (1H, brs, H-6), 3.26 (1H, m, H-15a), 3.22 (1H, m, H-22a), 2.82 (1H, d, J=7.6 Hz, H-17), 2.74 (1H, m, H-2a), 2.53 (1H, m, H-2b), 2.38 (1H, m, H-23a), 2.27 (1H, d, J=10.0 Hz, H-5), 2.13 (2H, m, H-7), 2.11 (1H, m, H-23b), 2.07 (1H, m, H-8), 2.04 (1H, m, H-1a), 2.04 (1H, m, H-11a), 1.84 (1H, m, H-12a), 1.76 (1H, m, H-22b), 1.75 (1H, m, H-15b), 1.67 (1H, m, H-12b), 1.59 (3H, s, H-18), 1.58 (3H, s, H-27), 1.52 (3H, s, H-29), 1.37 (3H, s, H-21), 1.34 (1H, m, H-1), 1.34 (3H, s, H-26), 1.11 (1H, m, H-11b), 0.78 (1H, d, J=4.1 Hz, H-19a), 0.44 (1H, d, J=4.1 Hz, H-19b) [7].

4: ¹H-NMR (400 MHz, pyridine-d₅) δ 4.96 (1H, m, H-16), 4.21 (1H, m, H-12), 4.07 (1H, t, J=10.0, 17.0 Hz, H-24), 3.99 (1H, t, J=5.0, 9.0 Hz, H-6), 3.80 (1H, m, H-3), 3.25 (1H, d, J=8.2 Hz, H-17), 2.42 (1H, m, H-11b), 1.94 (3H, s, H-21), 1.82 (3H, s, H-28), 1.62 (3H, s, H-18), 1.60 (3H, s, H-27), 1.57 (3H, s, H-26), 1.40 (3H, s, H-29), 1.37 (3H, s, H-30), 0.61 (1H, d, J=4.8 Hz, H-19a), 0.48 (1H, d, J=4.0 Hz, H-19b) [6].

5: LC-MS m/z 525.3201 ([M+Na]⁺) C₃₀H₄₆O₆Na=525.31921. ¹H-NMR (500 MHz, CD₃OD) δ 4.15 (1H, dd, J=9.3, 2.6 Hz, H-12), 3.87 (1H, dd, J=7.9, 6.4 Hz, H-24), 3.50 (1H, td, J=10.0, 3.5 Hz, H-6), 2.97 (1H, s, H-17), 2.65 (1H, m, H-2a), 2.63 (1H, m, H-11a), 2.49 (1H, m, H-2b), 2.35 (1H, d, J=18.0 Hz, H-15a), 2.24 (1H, m, H-22a), 2.15 (1H, m, H-1a), 2.08 (1H, d, J=18.0 Hz, H-15b), 1.98 (1H, d, J=9.9 Hz, H-5), 1.92 (1H, dd, J=11.2, 5.0 Hz, H-8), 1.86 (1H, ddd, J=13.0, 6.4, 3.9 Hz, H-23), 1.75 (1H, m, H-22b), 1.46 (1H, m, H-1b), 1.43 (2H, m, H-7), 1.34 (3H, s, H-18), 1.33 (3H, s, H-28), 1.24 (3H, s, H-21), 1.21 (3H, s, H-29), 1.18 (3H, s, H-27), 1.15 (3H, s, H-26), 1.14 (3H, s, H-30), 1.10 (1H, dd, J=11.2, 2.7 Hz, H-11b), 0.80 (1H, d, J=4.4 Hz, H-19a), 0.58 (1H, d, J=4.5 Hz, H-19b); ¹³C-NMR (125 MHz, CD₃OD) δ 219.4 (s, C-3), 217.7 (s, C-16), 85.3 (d, C-24), 85.2 (s, C-20), 73.1 (d, C-12), 72.2 (s, C-25), 69.8 (d, C-6), 69.4 (d, C-17), 54.0 (d, C-5), 52.7 (s, C-14), 51.4 (s, C-4), 51.4 (t, C-15), 46.9 (d, C-8), 44.4 (s, C-13), 42.8 (t, C-22), 38.1 (t, C-7), 36.6 (t, C-2), 36.6 (t, C-11), 32.6 (t, C-1), 32.0 (t, C-19), 30.0 (s, C-10), 28.3 (q, C-28), 26.4 (t, C-23), 26.2 (q, C-27), 25.9 (q, C-26), 22.4 (q, C-21), 22.2 (s, C-9), 20.6 (q, C-29), 19.8 (q, C-30), 14.6 (q, C-18).

6: ¹H-NMR (400 MHz, pyridine-d₅) δ 5.01 (1H, m, H-16), 4.26 (1H, brs, H-12), 4.07 (1H, m, H-24), 3.70 (1H, m, H-3), 3.31 (1H, d, J=8.0 Hz, H-17), 2.83 (1H, m, H-6), 2.66 (1H, m, H-22a), 2.44 (1H, dd, J=5.6, 14.8 Hz, H-11a), 2.35 (1H, m, H-23a), 2.24 (1H, m, H-15b), 2.12 (1H, m, H-11b), 2.12 (1H, m, H-23b), 2.05 (1H, m, H-1a), 2.01 (1H, m, H-1b), 2.00 (1H, m, H-22b), 1.99 (3H, s, H-28), 1.94 (1H, s, H-15b), 1.93 (1H, s, H-8), 1.88 (1H, m, H-7a), 1.87 (3H, s, H-21), 1.83 (3H, s, H-18), 1.81 (1H, m, H-7b), 1.79 (1H, d, J=10.0 Hz, H-5), 1.74 (1H, m, H-2a), 1.60 (3H, s, H-27), 1.42 (3H, s, H-26), 1.44 (3H, s, H-29), 1.41 (3H, s, H-30), 1.37 (1H, m, H-2b), 0.66 (1H, d, J=4.1 Hz, H-19a), 0.52 (1H, d, J=4.1 Hz, H-19b) [7].

7: ¹H-NMR (500 MHz, CD₃OD) δ 4.77 (1H, td, J=10.4, 2.6 Hz, H-6), 4.50 (1H, dd, J=7.0, 7.0, 7.7 Hz, H-16), 3.86 (1H, dd, J=8.8, 5.8 Hz, H-12), 3.84 (1H, dd, J=13.2, 6.2 Hz, H-24), 3.24 (1H, dd, J=10.4, 5.0 Hz, H-3), 2.67 (1H, d, J=8.1 Hz, H-17), 2.04 (1H, m, H-22a), 2.03 (1H, m, H-23a), 1.96 (1H, m, H-11a), 1.94 (1H, m, H-15a), 1.94 (1H, m, H-22b), 1.93 (1H, m, H-23b), 1.76 (1H, m, H-2a), 1.75 (1H, m, H-11b), 1.74 (1H, m, H-8), 1.72 (1H, d, J=10.5 Hz, H-5), 1.65 (1H, m, H-2b), 1.64 (1H, m, H-1a), 1.52 (1H, m, H-7a), 1.47 (3H, s, H-21), 1.45 (1H, m, H-15b), 1.41 (1H, m, H-7b), 1.32 (1H, m, H-1b), 1.29 (3H, s, H-18), 1.20 (3H, s, H-27), 1.15 (3H, s, H-26), 1.02 (3H, s, H-28), 1.02 (3H, s, H-30), 0.92 (3H, s, H-29); ¹³C-NMR (125 MHz, CD₃OD) δ 88.1 (s, C-20), 84.4 (d, C-24), 78.5 (d, C-3), 73.8 (d, C-12), 73.7 (d, C-6), 73.5 (d, C-16), 72.0 (s, C-25), 53.3 (d, C-17), 51.4 (d, C-5), 51.4 (s, C-14), 49.6 (t, C-15), 48.2 (d, C-8), 47.0 (s, C-13), 42.4 (s, C-4), 38.9 (t, C-22), 37.6 (t, C-11), 35.1 (t, C-7), 33.5 (t, C-1), 31.9 (t, C-2), 31.9 (t, C-19), 30.2 (s, C-10), 28.0 (q, C-28), 26.7 (q, C-26), 26.6 (q, C-27), 26.4 (q, C-21), 26.3 (t, C-23), 21.7 (s, C-9), 21.7 (q, C-30), 21.2 (q, C-18), 16.1 (q, C-29).

8: LC-MS m/z 511.3386 ([M+Na]+), m/z 527.3142 ([M+K]+), C₃₀H₄₈O₅Na=511.33994, C₃₀H₄₈O₅K=527.31388. ¹H-NMR (500 MHz, CD₃OD) δ 5.37 (1H, brs, H-1), 5.26 (1H, brs, H-11), 4.65 (1H, ddd, J=6.2, 7.7, 8.5 Hz, H-16), 4.18 (1H, m, H-6), 3.76 (1H, dd, J=8.3, 5.9 Hz, H-24), 3.40 (1H, t, J=4.6 Hz, H-3), 2.86 (1H, d, J=14.2 Hz, H-19a) 2.80 (1H, brs, J=14.6 Hz, H-19b), 2.60 (1H, m, H-22a), 2.29 (1H, d, J=7.7 Hz, H-17), 2.28 (1H, m, H-2a), 2.18 (1H, m, H-8), 2.07 (1H, m, H-2b), 2.03 (1H, m, H-12a), 2.03 (1H, m, H-23), 2.01 (1H, dd, J=13.1, 8.5 Hz, H-15a), 1.90 (1H, dd, J=16.8, 4.6 Hz, H-12b), 1.83 (1H, d, J=7.8 Hz, H-5), 1.72 (2H, m, H-7), 1.63 (1H, m, H-22b), 1.44 (1H, dd, J=12.8, 6.2 Hz, H-15b), 1.26 (3H, s, H-27), 1.22 (3H, s, H-21), 1.17 (3H, s, H-28), 1.12 (3H, s, H-26), 0.97 (3H, s, H-29), 0.96 (3H, s, H-18), 0.76 (3H, s, H-30); ¹³C-NMR (125 MHz, CD₃OD) δ 141.1 (s, C-9), 136.8 (s, C-10), 122.6 (d, C-1), 118.6 (d, C-11), 88.3 (s, C-20), 82.7 (d, C-24), 75.8 (d, C-3), 74.7 (d, C-6), 74.1 (d, C-16), 72.5 (s, C-25), 58.2 (d, C-5), 57.9 (d, C-17), 46.7 (s, C-13), 45.6 (t, C-19), 44.7 (s, C-14), 44.5 (t, C-15), 41.8 (d, C-8), 39.3 (t, C-7), 38.9 (t, C-12), 38.5 (s, C-4), 35.5 (t, C-22), 32.4 (t, C-2), 28.5 (q, C-21), 28.4 (q, C-29), 27.6 (q, C-27), 26.8 (t, C-23), 26.6 (q, C-26), 23.2 (q, C-28), 19.2 (q, C-18), 17.9 (q, C-30).

9: LC-MS m/z 505.3516 ([M+H]⁺) C₃₀H₄₉O₆=505.35291. ¹H-NMR (500 MHz, CD₃OD) δ 4.49 (1H, ddd, J=6.9, 7.7, 8.0 Hz, H-16), 3.91 (1H, m, H-24), 3.90 (1H, s, H-12), 3.44 (1H, m, H-6), 3.28 (1H, s, H-1), 3.26 (1H, dd, J=4.5, 11.5 Hz, H-3), 2.31 (1H, d, J=8.0 Hz, H-17), 2.08 (1H, m, H-23a), 2.07 (1H, m, H-15a), 2.04 (1H, m, H-22a), 1.97 (1H, m, H-11a), 1.97 (1H, m, H-23b), 1.83 (1H, m, H-22b), 1.74 (1H, m, H-2a), 1.68 (1H, m, H-2b), 1.58 (1H, m, H-8), 1.56 (1H, m, H-15b), 1.53 (3H, s, H-21), 1.50 (1H, d, J=9.8 Hz, H-5), 1.41 (2H, m, H-7), 1.32 (3H, s, H-18), 1.31 (1H, m, H-11b), 1.24 (3H, s, H-28), 1.18 (3H, s, H-26), 1.18 (3H, s, H-27), 1.13 (3H, s, H-30), 0.96 (3H, s, H-29), 0.89 (1H, d, J=4.1 Hz, H-19a), 0.65 (1H, d, J=3.6 Hz, H-19b); ¹³C-NMR (125 MHz, CD₃OD) δ 88.0 (s, C-20), 85.2 (d, C-24), 84.8 (d, C-12), 82.5 (d, C-1), 79.3 (d, C-3), 73.4 (d, C-16), 72.5 (s, C-25), 70.9 (d, C-6), 60.9 (d, C-17), 54.2 (d, C-5), 52.3 (s, C-14), 50.1 (d, C-8), 49.8 (t, C-15), 48.3 (s, C-13), 42.8 (s, C-4), 40.0 (t, C-7), 39.8 (t, C-22), 37.0 (t, H-19), 36.4 (t, C-11), 33.2 (s, C-10), 31.3 (t, C-2), 30.2 (s, C-9), 29.1 (q, C-28), 26.8 (q, C-26), 26.6 (t, C-23), 25.5 (q, C-21), 25.5 (q, C-27), 19.5 (q, C-30), 15.9 (q, C-29), 14.2 (q, C-18).

13: LC-MS m/z 527.3346 ([M+Na]⁺) C₃₀H₄₈O₆Na=527.33486. ¹H-NMR (500 MHz, CD₃OD) δ 5.31 (1H, s, H-11), 4.52 (1H, ddd, J=7.7, 6.9, 7.7 Hz, H-16), 4.14 (1H, brs, H-12), 3.93 (1H, td, J=0.8, 3.8 Hz, H-6), 3.90 (1H, dd, J=8.1, 5.3 Hz, H-24), 2.80 (1H, ddd, J=16.1, 10.9, 5.2 Hz, H-2a), 2.42 (1H, m, H-8), 2.39 (1H, m, H-2b), 2.31 (1H, d, J=7.9 Hz, H-17), 2.10 (2H, m, H-1), 2.09 (1H, m, H-23a), 2.05 (1H, dd, J=12.9, 8.0 Hz, H-15a), 2.00 (1H, m, H-22a), 1.97 (1H, m, H-23b), 1.88 (1H, m, H-22b), 1.81 (1H, dt, J=4.0, 12.0 Hz, H-7), 1.76 (1H, d, J=10.6 Hz, H-5), 1.64 (1H, dd, J=12.9, 5.7 Hz, H-15b), 1.51 (3H, s, H-21), 1.33 (3H, s, H-29), 1.33 (3H, s, H-28), 1.19 (3H, s, H-27), 1.17 (3H, s, H-26), 1.01 (1H, s, H-19), 1.00 (3H, s, H-18), 0.85 (3H, s, H-30); ¹³C-NMR (125 MHz, CD₃OD) δ 222.0 (s, C-3), 148.6 (s, C-9), 122.6 (d, C-11), 87.8 (s, C-20), 85.1 (d, C-24), 74.0 (d, C-12), 73.2 (d, C-16), 72.4 (s, C-25), 70.0 (d, C-6), 59.6 (d, C-17), 58.0 (d, C-5), 51.4 (s, C-14), 48.8 (s, C-4), 46.9 (t, C-15), 46.7 (s, C-13), 41.7 (d, C-8), 41.2 (s, C-10), 39.5 (t, C-22), 38.9 (t, C-7), 36.4 (t, C-1), 34.2 (t, C-2), 31.9 (q, C-28), 26.7 (t, C-23), 26.7 (q, C-26), 25.8 (q, C-21), 25.8 (q, C-27), 24.4 (q, C-19), 20.7 (q, C-29), 19.5 (q, C-30), 12.3 (q, C-18).

14: ¹H-NMR (400 MHz, pyridine-d₅) δ 5.62 (1H, s, H-11), 4.87 (1H, m, H-16), 4.49 (1H, s, H-12), 4.44 (1H, m, H-6), 4.09 (1H, m, H-24), 3.50 (1H, dd, J=4.8, 11.2 Hz, H-3), 2.72 (1H, d, J=10.4 Hz, H-8), 2.61 (1H, d, J=6.4 Hz, H-17), 2.27 (1H, m, H-22), 2.25 (1H, m, H-23a), 2.19 (1H, m, H-7a), 2.14 (1H, m, H-15a), 2.11 (1H, m, H-23b), 2.01 (1H, m, H-15b), 1.95 (3H, s, H-28), 1.94 (1H, m, H-2), 1.91 (3H, s, H-21), 1.84 (1H, m, H-1a), 1.82 (1H, m, H-7b), 1.62 (1H, m, H-1b), 1.49 (3H, s, H-26), 1.49 (3H, s, H-29), 1.47 (3H, s, H-18), 1.40 (3H, s, H-27), 1.36 (1H, m, H-5), 1.19 (3H, s, H-19), 0.92 (3H, s, H-30) [8].

15: HRTOFMS m/z 545.34499 ([M+Na]+) C₃₀H₅₀O₇Na=545.34542. ¹H-NMR (400 MHz, CD₃OD) δ 4.66 (1H, ddd, J=6.8, 7.6, 7.6 Hz, H-16), 4.10 (1H, td, J=4.1, 11.0 Hz, H-6), 4.01 (1H, d, J=11.0 Hz, H-19a), 3.88 (1H, d, J=11.0 Hz, H-19b), 3.76 (1H, m, H-24), 3.22 (1H, d, J=6.0 Hz, H-11), 3.08 (1H, dd, J=11.3, 5.1 Hz, H-3), 2.59 (1H, m, H-22a), 2.51 (1H, dd, J=13.5, 4.1 Hz, H-8), 2.30 (1H, d, J=7.6 Hz, H-17), 2.02 (2H, m, H-23), 1.98 (1H, m, H-12a), 1.88 (1H, m, H-15a), 1.84 (1H, m, H-7a), 1.81 (1H, m, H-12b), 1.69 (1H, m, H-7b), 1.63 (1H, m, H-1a), 1.61 (1H, m, H-22b), 1.60 (1H, m, H-2a), 1.54 (1H, m, H-2b), 1.42 (1H, m, H-5), 1.42 (1H, m, H-15b), 1.36 (3H, s, H-21), 1.31 (3H, s, H-28), 1.25 (3H, s, H-27), 1.24 (3H, s, H-18), 1.12 (3H, s, H-26), 1.04 (1H, m, H-1b), 0.99 (3H, s, H-30), 0.95 (3H, s, H-29); ¹³C-NMR (100 MHz, CD₃OD) δ 88.2 (s, C-20), 82.6 (d, C-24), 79.3 (d, C-3), 74.6 (d, C-16), 72.5 (s, C-25), 69.8 (d, C-6), 67.5 (s, C-9), 61.1 (t, C-19), 57.9 (d, C-11), 57.6 (d, C-17), 56.8 (d, C-5), 46.8 (s, C-10), 46.3 (t, C-15), 45.5 (s, C-13), 45.2 (s, C-14), 40.4 (s, C-4), 38.9 (t, C-7), 37.9 (d, C-8), 36.6 (t, C-22), 36.5 (t, C-12), 35.6 (q, C-21), 31.7 (q, C-28), 27.6 (q, C-27), 27.2 (t, C-2), 26.8 (t, C-23), 26.6 (q, C-26), 23.9 (t, C-1), 20.2 (q, C-18), 20.0 (q, C-30), 16.7 (q, C-29).

16: ¹H-NMR (400 MHz, pyridine-d₅) δ 5.78 (1H, d, J=5.72 Hz, H-11), 5.03 (1H, m, H-16), 4.46 (1H, m, H-6), 4.24 (1H, m, H-12), 3.93 (1H, m, H-24), 3.49 (1H, m, H-3), 3.39 (1H, d, J=8.0 Hz, H-17), 2.93 (1H, m, H-22a), 2.68 (1H, dd, J=7.6, 12.4 Hz, H-8), 2.31 (1H, m, H-23a), 2.22 (1H, m, H-7a), 2.18 (1H, dd, J=8.0, 12.4 Hz, H-15a), 1.97 (1H, m, H-1), 1.96 (3H, s, H-28), 1.95 (1H, m, H-7b), 1.95 (1H, m, H-23b), 1.94 (1H, m, H-15b), 1.86 (1H, m, H-22b), 1.82 (1H, m, H-2a), 1.76 (3H, s, H-21), 1.60 (1H, m, H-2b), 1.57 (3H, s, H-27), 1.54 (3H, s, H-29), 1.41 (1H, d, J=10.4 Hz, H-5), 1.32 (3H, s, H-26), 1.29 (3H, s, H-30), 1.23 (3H, s, H-18), 1.20 (3H, s, H-19) [9].

17: HRTOFMS m/z 527.33503 ([M+Na]⁺) C₃₀H₄₈O₆Na=527.33486. ¹H-NMR (400 MHz, CD₃OD) δ 5.55 (1H, d, J=2.3 Hz, H-11), 4.66 (1H, ddd, J=8.0, 8.0, 7.5 Hz, H-16), 4.20 (1H, td, J=10.7, 4.1 Hz, H-6), 3.77 (1H, t, J=7.6 Hz, H-24), 3.12 (1H, dd, J=9.5, 6.1 Hz, H-3), 2.99 (1H, m, H-8), 2.79 (1H, d, J=8.4 Hz, H-17), 2.45 (1H, m, H-22a), 2.08 (1H, m, H-15a), 1.99 (1H, m, H-7a), 1.98 (1H, m, H-1a), 1.97 (1H, m, H-23), 1.80 (1H, m, H-15b), 1.70 (1H, m, H-2), 1.64 (1H, m, H-22b), 1.54 (1H, m, H-7b), 1.50 (1H, m, H-1b), 1.36 (3H, s, H-21), 1.30 (3H, s, H-28), 1.28 (3H, s, H-18), 1.24 (3H, s, H-27), 1.21 (3H, s, H-19), 1.12 (3H, s, H-26), 1.08 (1H, m, H-5), 1.05 (3H, s, H-29), 0.79 (3H, s, H-30); ¹³C-NMR (100 MHz, CD₃OD) δ 207 (s, C-12), 168.2 (s, C-9), 119.8 (d, C-11), 88.15 (s, C-20), 83.3 (d, C-24), 79.1 (d, C-3), 74.0 (d, C-16), 72.0 (s, C-25), 69.4 (d, C-6), 59.2 (s, C-13), 57.4 (d, C-5), 51.0 (d, C-17), 44.3 (t, C-15), 43.5 (d, C-8), 43.5 (s, C-14), 42.8 (s, C-10), 40.8 (s, C-4), 39.0 (t, C-7), 37.1 (t, C-1), 36.6 (t, C-22), 31.5 (q, C-28), 30.9 (q, C-21), 27.8 (t, C-2), 27.4 (q, C-27), 26.7 (q, C-26), 26.0 (t, C-23), 23.4 (q, C-19), 19.7 (q, C-30), 17.7 (q, C-18), 16.5 (q, C-29)

18: ¹H-NMR (400 MHz, CD₃OD) δ 5.32 (1H, s, H-12), 5.13 (1H, s, H-11), 4.63 (1H, m, H-16), 4.10 (1H, td, J=10.8, 4.0 Hz, H-6), 3.75 (1H, dd, J=8.7, 5.3 Hz, H-24), 3.10 (1H, dd, J=9.0, 7.1 Hz, H-3), 2.56 (1H, m, H-8), 2.45 (1H, d, J=7.2 Hz, H-17), 2.08 (1H, m, H-23a), 1.99 (1H, m, H-15a), 1.94 (1H, m, H-23b), 1.84 (1H, m, H-7a), 1.74 (1H, m, H-22), 1.72 (1H, m, H-1a), 1.67 (1H, m, H-2), 1.61 (1H, dd, J=13.0, 5.7 Hz, H-15b), 1.46 (1H, m, H-7b), 1.43 (1H, m, H-1b), 1.28 (3H, s, H-28), 1.23 (3H, s, H-21), 1.21 (3H, s, H-27), 1.12 (3H, s, H-26), 1.11 (3H, s, H-19), 1.08 (3H, s, H-18), 1.02 (3H, s, H-29), 1.01 (1H, m, H-5), 0.94 (3H, s, H-30); ¹³C-NMR (100 MHz, CD₃OD) δ 151.5 (s, C-9), 117.4 (d, C-11), 87.7 (s, C-20), 83.3 (d, C-24), 79.5 (d, C-3), 79.4 (d, C-12), 73.8 (d, C-16), 72.7 (s, C-25), 70.1 (d, C-6), 58.6 (d, C-17), 58.1 (d, C-5), 49.1 (s, C-13), 47.6 (s, C-14), 45.2 (t, C-15), 42.1 (s, C-10), 41.9 (d, C-8), 40.6 (s, C-4), 39.4 (t, C-7), 37.4 (t, C-1), 36.3 (t, C-22), 31.7 (q, C-28), 27.6 (t, C-2), 27.1 (q, C-27), 26.8 (t, C-23), 26.2 (q, C-26), 23.7 (q, C-21), 23.4 (q, C-19), 19.7 (q, C-30), 16.4 (q, C-29), 13.5 (q, C-18).

22: HRTOFMS m/z 531.3664 ([M+Na]⁺) C₃₀H₅₂O₆Na=531.36616. ¹H-NMR (400 MHz, CD₃OD) δ 4.46 (1H, ddd, J=5.4, 7.9, 7.9 Hz, H-16), 3.82 (1H, dd, J=3.9, 5.9 Hz, H-12), 3.41 (1H, m, H-6), 3.40 (1H, m, H-24), 3.23 (1H, dd, J=10.8, 4.6 Hz, H-3), 2.30 (1H, dd, J=11.0, 7.8 Hz, H-17), 2.02 (1H, m, H-15a), 2.00 (1H, dd, J=13.3, 5.1 Hz, H-11a), 1.92 (1H, m, H-20), 1.82 (1H, m, H-22a), 1.75 (1H, dd, J=9.3, 13.3 Hz, H-11b), 1.74 (1H, m, H-2a), 1.66 (1H, m, H-1a), 1.64 (1H, m, H-8), 1.62 (1H, m, H-2b), 1.61 (1H, m, H-23a), 1.43 (1H, m, H-22b), 1.41 (1H, m, H-7a), 1.41 (1H, m, H-15b), 1.35 (1H, m, H-7b), 1.34 (1H, m, H-5), 1.29 (1H, m, H-23b), 1.28 (1H, m, H-1b), 1.24 (3H, s, H-28), 1.10 (3H, s, H-18), 1.17 (3H, s, H-27), 1.15 (3H, s, H-26), 1.07 (3H, d, J=6.6 Hz, H-21), 1.02 (3H, s, H-30), 0.95 (3H, s, H-29), 0.50 (1H, d, J=4.5 Hz, H-19a), 0.47 (1H, d, J=4.5 Hz, H-19b); ¹³C-NMR (100 MHz, CD₃OD) δ 79.4 (d, C-3, 78.3 (d, C-24), 73.8 (s, C-25), 73.7 (d, C-12), 73.2 (d, C-16), 70.3 (d, C-6), 54.6 (d, C-5), 50.5 (s, C-13) 50.2 (t, C-15), 49.8 (d, C-17), 49.6 (d, C-8), 47.3 (s, C-14), 42.7 (s, C-4), 39.7 (t, C-11), 39.3 (t, C-7), 33.8 (t, C-22), 33.7 (t, C-1), 32.3 (t, C-19), 31.1 (t, C-2), 30.7 (s, C-10), 29.4 (d, C-20), 29.0 (q, C-28), 28.5 (t, C-23), 25.5 (q, C-27), 25.4 (q, C-26), 22.0 (s, C-9), 21.9 (q, C-30), 18.5 (q, C-18), 16.0 (q, C-21), 16.0 (q, C-29).

23: HRTOFMS m/z 531.3672 ([M+Na]⁺) C₃₀H₅₂O₆Na=531.36616. ¹H-NMR (400 MHz, CD₃OD) δ 4.19 (1H, dd, J=5.8, 8.2 Hz, H-16), 3.44 (1H, m, H-6), 3.40 (1H, m, H-24), 3.22 (1H, dd, J=10.9, 4.4 Hz, H-3), 2.15 (1H, dd, J=13.2, 8.5 Hz, H-15a), 2.11 (1H, m, H-12a), 2.09 (1H, m, H-11a), 2.04 (1H, m, H-20), 1.78 (1H, m, H-8), 1.71 (1H, m, H-2a), 1.71 (1H, m, H-22a), 1.62 (1H, m, H-23a), 1.61 (1H, m, H-2b), 1.57 (1H, m, H-1a), 1.44 (1H, m, H-12b), 1.41 (1H, m, H-7a), 1.41 (1H, m, H-15b), 1.41 (1H, m, H-23b), 1.35 (1H, m, H-7b), 1.34 (1H, d, J=9.9 Hz, H-5), 1.29 (1H, m, H-22b), 1.28 (3H, s, H-30), 1.25 (1H, m, H-1b), 1.24 (3H, s, H-28), 1.23 (3H, s, H-18), 1.16 (1H, m, H-11b), 1.16 (3H, s, H-27), 1.15 (3H, s, H-26), 0.96 (3H, s, H-29), 0.51 (1H, d, J=4.2 Hz, H-19a), 0.43 (1H, d, J=4.2 Hz, H-19b); ¹³C-NMR (100 MHz, CD₃OD) δ 89.3 (d, C-17), 82.5 (d, C-16), 79.4 (d, C-3), 78.0 (d, C-24), 73.8 (s, C-25), 70.3 (d, C-6), 54.7 (d, C-5), 51.6 (s, C-13), 51.0 (d, C-8), 49.3 (t, C-15), 47.1 (s, C-14), 42.7 (s, C-4), 39.3 (t, C-7), 35.7 (d, C-20), 33.5 (t, C-1), 32.6 (t, C-19), 31.1 (t, C-2), 31.1 (s, C-10), 30.5 (t, C-22), 29.2 (t, C-23), 29.0 (q, C-28), 28.0 (t, C-12), 27.4 (t, C-11), 25.4 (q, C-26), 25.4 (q, C-27), 23.0 (q, C-30), 22.2 (s, C-9), 21.0 (q, C-18), 15.9 (q, C-29), 14.2 (q, C-21).

24: HRTOFMS m/z 525.32063 ([M+Na]⁺) C₃₀H₄₆O₆Na=525.31921. ¹H-NMR (400 MHz, CD₃OD) δ 5.68 (1H, d, J=5.5 Hz, H-11), 5.46 (1H, dd, J=3.5, 1.3 Hz, H-7), 4.54 (1H, dd, J=10.3, 3.4 Hz, H-6), 4.03 (1H, d, J=6.0 Hz, H-12), 3.73 (1H, d, J=5.6 Hz, H-3), 3.30 (1H, m, H-24), 3.04 (1H, d, J=14.0 Hz, H-19a), 2.94 (1H, d, J=7.3 Hz, H-17), 2.67 (1H, d, J=14.3 Hz, H-19b), 2.45 (1H, d, J=17.6 H-15a), 2.10 (1H, d, J=17.7 H-15b), 2.00 (1H, m, H-2a), 1.90 (1H, m, H-22a), 1.74 (1H, m, H-2b), 1.73 (1H, m, H-20), 1.61 (1H, m, H-5), 1.59 (1H, m, H-23a), 1.58 (1H, m, H-1a), 1.56 (1H, m, H-22b), 1.41 (1H, m, H-23b), 1.38 (1H, m, H-1b), 1.32 (3H, s, H-30), 1.22 (3H, s, H-28), 1.17 (3H, s, H-27), 1.15 (3H, s, H-26), 1.12 (3H, s, H-29), 1.09 (3H, d, J=6.8 Hz, H-21), 0.84 (1H, s, H-18); ¹³C-NMR (100 MHz, CD₃OD) δ 221.4 (s, C-16), 141.8 (s, C-8), 136.8 (d, C-7), 134.2 (s, C-9), 128.4 (d, C-11), 88.3 (s, C-10), 86.8 (d, C-3), 79.5 (d, C-24), 73.8 (s, C-25), 72.3 (d, C-12), 68.9 (d, C-6), 63.6 (d, C-5), 57.2 (d, C-17), 49.8 (t, C-15), 48.7 (s, C-13), 48.3 (s, C-4), 46.9 (s, C-14), 41.5 (t, C-19), 38.8 (t, C-1), 33.6 (t, C-22), 32.8 (d, C-20), 29.9 (t, C-23), 28.7 (q, C-30), 25.8 (t, C-2), 25.6 (q, C-27), 25.3 (q, C-29), 25.1 (q, C-26), 23.4 (q, C-28), 18.6 (q, C-18), 18.5 (q, C-21).

25: HRTOFMS m/z 525.3566 ([M+Na]⁺) C₃₁H₅₀O₅Na=525.35559. ¹H-NMR (400 MHz, CD₃OD) δ 5.47 (1H, brs, H-11), 5.36 (1H, brs, H-7), 4.55 (1H, ddd, J=5.6, 7.7, 8.0 Hz, H-16), 4.05 (1H, dd, J=10.3, 2.8 Hz, H-6), 3.72 (1H, d, J=5.6 Hz, H-3), 3.38 (1H, dd, J=10.9, 1.9 Hz, H-24), 3.33 (3H, s, H-1′), 3.02 (1H, d, J=13.6 Hz, H-19a), 2.55 (1H, d, J=13.9 Hz, H-19b), 2.18 (2H, brs, H-12), 2.10 (1H, dd, J=12.9, 8.1 Hz, H-15a), 1.97 (1H, m, H-2a), 1.94 (1H, m, H-20), 1.80 (1H, m, H-22a), 1.74 (1H, m, H-15b), 1.72 (1H, m, H-2b), 1.70 (1H, m, H-17), 1.63 (1H, m, H-23a), 1.57 (1H, d, J=10.6 Hz, H-5), 1.49 (1H, m, H-1a), 1.43 (1H, m, H-23b), 1.32 (1H, m, H-1b), 1.23 (1H, m, H-22b), 1.17 (3H, s, H-27), 1.15 (3H, s, H-26), 1.15 (3H, s, H-28), 1.07 (3H, s, H-29), 0.97 (3H, d, J=6.4 Hz, H-21), 0.97 (3H, s, H-30), 0.87 (3H, s, H-18); ¹³C-NMR (100 MHz, CD₃OD) δ 145.7 (s, C-8), 132.8 (s, C-9), 130.5 (d, C-7), 127.6 (d, C-11), 88.6 (s, C-10), 86.8 (d, C-3), 79.2 (d, C-6), 78.5 (d, C-24), 73.8 (s, C-25), 72.8 (d, C-16), 62.8 (d, C-5), 57.0 (d, C-17), 56.5 (q, C-1′), 50.9 (s, C-14), 48.0 (s, C-4), 45.3 (t, C-15), 45.0 (t, C-13), 41.4 (t, C-19), 39.3 (t, C-12), 38.5 (t, C-1), 33.8 (t, C-22), 30.0 (d, C-20), 28.5 (t, C-23), 26.1 (q, C-30), 25.7 (t, C-2), 25.4 (q, C-26), 25.4 (q, C-27), 25.4 (q, C-28), 25.2 (q, C-29), 18.6 (q, C-21), 17.2 (q, C-18).

26: HRTOFMS m/z 529.35228 ([M+Na]⁺) C₃₀H₅₀O₆Na=529.35051. ¹H-NMR (400 MHz, CD₃OD) δ 3.76 (1H, ddd, J=11.6, 7.2, 4.5 Hz, H-6), 3.51 (1H, dd, J=10.7, 5.0 Hz, H-19a), 3.44 (1H, dd, J=11.1 Hz, H-19b), 3.36 (1H, dd, J=4.5, 11.2 Hz, H-3), 3.28 (1H, m, H-24), 3.04 (1H, m, H-11), 2.74 (1H, m, H-22a), 2.45 (1H, d, J=10.9 Hz, H-8), 2.37 (1H, d, J=7.1 Hz, H-17), 2.31 (1H, d, J=14.4 Hz, H-1a), 2.19 (1H, d, J=18.4 Hz, H-15a), 1.97 (1H, d, J=19.7 Hz, H-15b), 1.88 (1H, m, H-2a), 1.86 (1H, m, H-12a), 1.83 (1H, m, H-1b), 1.80 (1H, m, H-5), 1.78 (1H, m, H-22b), 1.76 (1H, m, H-20), 1.58 (1H, m, H-23a), 1.57 (1H, m, H-7a), 1.51 (1H, m, H-12b), 1.47 (1H, m, H-2b), 1.39 (1H, m, H-23b), 1.35 (1H, m, H-7b), 1.17 (3H, s, H-27), 1.17 (3H, s, H-28), 1.14 (3H, s, H-26), 1.07 (3H, s, H-18), 1.06 (3H, d, J=6.8 Hz, H-21), 0.96 (3H, s, H-30), 0.68 (3H, s, H-29); ¹³C-NMR (100 MHz, CD₃OD) δ 221.8 (s, C-16), 136.6 (s, C-10), 132.3 (s, C-9), 79.4 (d, C-24), 78.4 (d, C-3), 73.8 (s, C-25), 68.8 (t, C-19), 68.5 (t, C-6), 63.2 (d, C-179, 57.8 (d, C-5), 49.8 (t, C-15), 46.5 (s, C-14), 42.7 (s, C-13), 42.5 (s, C-4), 40.9 (d, C-8), 39.3 (d, C-11), 37.4 (t, C-7), 33.7 (t, C-12), 32.8 (t, C-2), 32.7 (d, C-20), 31.9 (t, C-1), 29.9 (t, C-23), 29.4 (t, C-22), 26.5 (q, C-28), 25.6 (q, C-27), 25.1 (q, C-26), 19.5 (q, C-21), 18.1 (q, C-18), 18.1 (q, C-30), 14.8 (q, C-29).

27: HRTOFMS m/z 531.36795 ([M+Na]⁺) C₃₀H₅₂O₆Na=531.36616. ¹H-NMR (400 MHz, CD₃OD) δ 4.46 (1H, ddd, J=5.6, 7.6, 7.6 Hz, H-16), 4.04 (1H, dd, J=7.3, 5.8 Hz, H-22), 3.53 (1H, dd, J=10.5, 2.2 Hz, H-24), 3.45 (1H, dt, J=9.8, 3.3 Hz, H-6), 3.32 (1H, dd, J=10.9, 4.4 Hz, H-3), 2.11 (1H, m, H-20), 2.08 (1H, m, H-17), 2.04 (1H, m, H-15a), 1.99 (1H, m, H-11a), 1.81 (1H, dd, J=12.1, 3.9 Hz, H-8), 1.77 (1H, m, H-23a), 1.70 (1H, m, H-2a), 1.65 (2H, m, H-12), 1.61 (1H, m, H-2b), 1.58 (1H, m, H-1a), 1.58 (1H, m, H-23b), 1.46 (1H, m, H-7a), 1.43 (1H, m, H-15b), 1.38 (1H, m, H-7b), 1.33 (1H, m, H-5), 1.26 (1H, m, H-1b), 1.22 (3H, s, H-27), 1.22 (3H, s, H-28), 1.19 (1H, m, H-11b), 1.18 (3H, s, H-18), 1.16 (3H, s, H-26), 0.99 (3H, s, H-30), 0.95 (3H, s, H-29), 0.92 (3H, d, J=6.4 Hz, H-21), 0.53 (1H, d, J=4.1 Hz, H-19a), 0.38 (1H, d, J=4.3 Hz, H-19b); ¹³C-NMR (100 MHz, CD₃OD) δ 79.5 (d, C-3), 79.1 (d, C-24), 75.3 (d, C-22), 73.5 (s, C-25), 73.1 (d, C-16), 69.8 (d, C-6), 54.5 (d, C-59, 53.3 (d, C-17), 48.8 (d, C-8), 48.5 (t, C-15), 47.8 (s, C-13), 46.5 (s, C-14), 42.7 (s, C-4), 38.9 (t, C-7), 35.4 (d, C-20), 34.9 (t, C-23), 34.0 (t, C-12), 33.5 (t, C-1), 31.8 (t, C-19), 31.1 (t, C-2), 30.7 (s, C-10), 28.8 (q, C-28), 27.0 (t, C-11), 25.6 (q, C-27), 25.1 (q, C-26), 22.2 (q, C-9), 20.5 (q, C-30), 19.2 (q, C-18), 15.8 (q, C-29), 13.4 (q, C-21).

28: HRTOFMS m/z 528.34288 ([M+Na]⁺) C₃₀H₄₈O₆Na=527.33486. ¹H-NMR (400 MHz, CD₃OD) δ 3.83 (1H, d, J=7.4 Hz, H-3), 3.76 (1H, d, J=7.4, 11.4 Hz, H-30a), 3.69 (1H, td, J=10.1, 2.7 Hz, H-6), 3.50 (1H, d, J=11.4 Hz, H-30b), 3.36 (1H, m, H-24), 2.75 (1H, d, J=14.8 Hz, H-19a), 2.57 (1H, m, H-7a), 2.46 (2H, s, H-15), 2.29 (1H, d, J=14.9 Hz, H-19a), 2.27 (1H, m, H-11a), 2.21 (1H, m, H-7b), 2.20 (1H, m, H-11b), 2.20 (1H, m, H-12a), 2.05 (1H, m, H-2a), 1.95 (1H, m, H-12b), 1.92 (1H, m, H-22a), 1.84 (1H, m, H-20), 1.76 (1H, m, H-1a), 1.67 (1H, m, H-2b), 1.64 (1H, m, H-23a), 1.59 (1H, m, H-22b), 1.50 (1H, d, J=9.6 Hz, H-5), 1.45 (1H, m, H-1b), 1.45 (1H, m, H-23b), 1.24 (3H, s, H-27), 1.21 (3H, s, H-26), 1.20 (3H, s, H-28), 1.18 (3H, s, H-29), 1.09 (3H, d, J=6.8 Hz, H-21), 1.07 (3H, s, H-18); ¹³C-NMR (100 MHz, CD₃OD) δ 221.9 (s, C-16), 132.5 (s, C-8), 131.2 (s, C-9), 89.1 (s, C-10), 87.9 (d, C-3), 79.5 (d, C-24), 73.8 (s, C-25), 68.7 (t, C-30), 68.2 (d, C-6), 64.9 (d, C-5), 62.7 (d, C-17), 51.0 (s, C-14), 47.0 (s, C-13), 46.5 (s, C-4), 42.1 (t, C-15), 40.7 (t, C-7), 40.3 (t, C-1), 39.1 (t, C-19), 33.9 (t, C-22), 33.0 (d, C-20), 30.82 (t, C-12), 30.76 (t, C-11), 30.0 (t, C-23), 27.2 (q, C-29), 26.2 (t, C-2), 25.6 (q, C-27), 25.1 (q, C-26), 24.9 (q, C-28), 19.6 (q, C-21), 19.2 (q, C-18).

Telomerase activation tests were performed on selected molecules as follows; For culturing HEKn (Human Primer Epidermal Keratinocyte, ATCC; PCS-200-010) cells, the cell medium containing the components given in Table 2 is prepared and the cells are grown in the Dermal Cell Basal medium (PCS-200-030) supplemented with components in 5% CO₂ environment at 37° C.

TABLE 2
Keratinocyte growth kit components (PCS-200-040).

| Component | Volume | Final Concentration | |
|---|---|---|---|
| Bovine Pituitary Extract (BPE) | 2 ml | 0.4% | |
| Rh-TGF-α | 0.5 ml | 0.5 | ng/ml |
| l-Glutamine | 15 ml | 6 | mM |
| Hyrocortisone Hemisuccinate | 0.5 ml | 100 | ng/ml |
| Epinephrine | 0.5 ml | 1 | mM |
| Rh Insulin | 0.5 ml | 5 | mg/ml |
| Apo-Transferrin | 0.5 ml | 1 | mM |

Telomerase activation was performed using the TELOTAGGG PCR ELISAPLUS kit (Roche; 12013789001, 16× version), a highly sensitive and quantitative method, according to the manufacturer's protocol, as follows:

After application of the selected molecules to HEKn cell lines at the defined dose interval and completion of 24-hour incubation, cells are collected and counted by hemocytometer. 2×10⁵ cells are transferred to clean microcentrifuge tubes and then centrifuged at 3000×g for 5 min (at 4° C.). The supernatant is removed and the cells in the pellet are suspended with 200 μl of lysis buffer (Solution 1) and incubated on ice for 30 min. After incubation the lysates are centrifuged at 16,000×g for 20 min (at 4° C.) and the cooled supernatant is transferred to clean microcentrifuge tubes.

PCR is designed for sample group and control group. 25 μl reaction mixture (Solution 2) and 5 μl internal standard solution (Solution 3) are transferred to the PCR tubes for both the positive and negative sample as well as for the sample to be investigated for activation or the prepared master mixture is taken to a 30 μl PCR tube to contain this content. For the samples to be tested, 2 μl of each PCR sample is added from the cell lysate. For the control group, 1 μl of the low or high concentration TS8 control sample (Solution 4 or 5) is transferred to a separate PCR tube. From the lysis buffer (Solution 1), 1 μl is transferred to a separate PCR tube.

TABLE 3
PCR program.

| | Time | | Temperature | Loop |
|---|---|---|---|---|
| Primer Elongation | 10-30 | dk | 25° C. | 1 |
| Telomerase inactivation | 5 | dk | 94° C. | 1 |
| Amplification: | | | | |
| Denaturation | 30 | s | 94° C. | |
| Annealing | 30 | s | 50° C. | 30 |
| Polymerization | 90 | s | 72° C. | |
| Final extension | 10 | dk | 72° C. | 1 |
| Final temperature | | | 4° C. | |

For each sample, 10 μl of the denaturing agent (Solution 7) is added to the nuclease-free 96-well plate. 2.5 μl of the amplified product is transferred to the plate and incubated for 10 min at +15 and +25° C. 100 μL Hybridization Buffer T (Solution 8) is added to a portion of the samples, 100 μL Hybridization Buffer IS (Solution 9) is added to the other portion. After homogenization, 100 μl of the reaction mixture is transferred to the MP module coated with streptavidin and the MP module is carefully closed with the special coating paper. After completion of the transfer process, the MP module is shaken at 300 rpm for 2 hours at 37° C. After incubation, the hybridization solutions are removed. Each well is washed 3 times with 250 μl of 1× wash buffer (Solution 10). The prepared anti-DIG-HRP working solution (Solution 11) is added to each of the wells. The MP module is sealed with special coating paper and incubated for 30 min at 15-25° C. with shaking at 300 rpm and the solution is carefully removed from the wells. Each well is washed 5 times in 1× wash buffer (Solution 10). From room temperature TMB substrate solution (Solution 13), 100 μl is added to each well. The MP module is covered with special coating paper and incubated at 15-25° C. for 10-20 minutes at 300 rpm with shaking. The color change is terminated by adding the post-incubation termination agent (Solution 14). The samples are measured at 690 nm with a reference wavelength of 450 nm in a microplate reader for 30 min. Compounds showing telomerase activation in the 1.5-fold and higher levels according to DMSO used as a negative control were considered active.

TABLE 4
Results of telomerase enzyme activation of the molecules

| | | |
|---|---|---|
| Molecules in the | | Fold increase over |
| TELOTAGGG PCR ELISA | | negative control |
| study | Doses | (DMSO) |

| 1 | 30 | nM | 1.73 |
|---|---|---|---|
| | 100 | nM | 0.72 |
| | 300 | nM | 0.74 |
| | 1000 | nM | 1.2 |
| 2 | 30 | nM | 1.05 |
| | 100 | nM | 0.43 |
| | 300 | nM | 0.74 |
| | 1000 | nM | 0.53 |
| 3 | 30 | nM | 2.36 |
| | 100 | nM | 0.78 |
| | 300 | nM | 1.24 |
| | 1000 | nM | 1.07 |
| 4 | 2 | nM | 2.13 |
| | 10 | nM | 2.22 |
| | 50 | nM | 2.4 |
| | 300 | nM | 2.13 |
| 5 | NT | | NT |
| 6 | 0.5 | nM | 9.23 |
| | 2 | nM | 10 |
| | 10 | nM | 6.38 |
| | 30 | nM | 2.37 |
| 7 | NT | | NT |
| 8 | NT | | NT |
| 9 | NT | | NT |
| 10 | 0.5 | nM | 8.43 |
| | 2 | nM | 9.35 |
| | 10 | nM | 9.95 |
| | 30 | nM | 4.6 |
| 11 | 0.5 | nM | 2.08 |
| | 2 | nM | 1.9 |
| | 10 | nM | 1.73 |
| | 30 | nM | 3.17 |
| 12 | 0.5 | nM | 4.65 |
| | 2 | nM | 2.14 |
| | 10 | nM | 1.78 |
| | 30 | nM | 0.84 |
| 13 | 30 | nM | 0.81 |
| | 100 | nM | 1.03 |
| | 300 | nM | 0.88 |
| | 1000 | nM | 0.78 |
| 14 | 2 | nM | 2.53 |
| | 10 | nM | 1.62 |
| | 50 | nM | 1.6 |
| | 300 | nM | 0.53 |
| 15 | 30 | nM | 0.82 |
| | 100 | nM | 1.1 |
| | 300 | nM | 1.06 |
| | 1000 | nM | 0.78 |
| 16 | 0.1 | nM | 0.98 |
| | 0.5 | nM | 2.31 |
| | 2 | nM | 2.78 |
| | 10 | nM | 0.95 |
| 17 | 0.5 | nM | 0.42 |
| | 2 | nM | 1.33 |
| | 10 | nM | 1 |
| | 50 | nM | 1.57 |
| 18 | 30 | nM | 0.95 |
| | 100 | nM | 0.46 |
| | 300 | nM | 1.1 |
| | 1000 | nM | 0.34 |
| 19 | 0.5 | nM | 2.65 |
| | 2 | nM | 1.24 |
| | 10 | nM | 11.32 |
| | 30 | nM | 1.93 |
| 20 | 0.5 | nM | 9.01 |
| | 2 | nM | 5.23 |
| | 10 | nM | 6.04 |
| | 30 | nM | 7.86 |
| 21 | 0.5 | nM | 9.25 |
| | 2 | nM | 1.43 |
| | 10 | nM | 1.04 |
| | 30 | nM | 1.18 |
| 22 | NT | | NT |
| 23 | 30 | nM | 0.58 |
| | 100 | nM | 0.83 |
| | 300 | nM | 0.34 |
| | 1000 | nM | 0.92 |
| 24 | NT | | NT |
| 25 | NT | | NT |
| 26 | 30 | nM | 0.74 |
| | 100 | nM | 0.94 |
| | 300 | nM | 0.92 |
| | 1000 | nM | 0.46 |
| 27 | 30 | nM | 0.64 |
| | 100 | nM | 0.63 |
| | 300 | nM | 0.65 |
| | 1000 | nM | 0.43 |
| 28 | 30 | nM | 0.92 |
| | 100 | nM | 0.88 |
| | 300 | nM | 0.85 |
| | 1000 | nM | 0.92 |

*NT = Not Tested.

In the obtained data, it can be seen that the saponin derivatives (Steroidal and/or Triterpenic) carrying the —OH group on carbon number 12 and/or modification in A ring are highly likely to increase telomerase activity in cells. Therefore, these molecules have the potential to be used in diseases/conditions that can be treated/ameliorated by telomerase activation associated with telomerase shortening. The molecular skeletons given above in formulations (29-59) are derived from this judgement.

What is claimed is:

1. A method of producing a telomerase activator which results in the production of new/novel molecules which can be used in diseases and/or conditions which can be treated/ameliorated by telomerase activation by carrying out microbial biotransformation with endophytic fungi on saponins from triterpenes group comprising the following steps,
washing plant materials with tap water and washing the plant materials with distilled water after cutting the plant materials to 3-5 cm length for surface sterilization,
keeping the washed plant materials in ethanol and then keeping the plant materials in sodium hypochlorite (NaOCl),
after being kept in sodium hypochlorite, washing the plant materials in ethanol and then in a container containing sterile water,
drying the plant materials in a laminar flow chamber and removal of outer shells of the plant materials under aseptic conditions,
cutting of internal tissues of the plant materials into small pieces and placing the internal tissues in petri dishes containing a medium and leaving the internal tissues to incubate,
transferring 100 μl of the water used in the final wash step of the plant materials into nutrient medium in a petri dish and spreading by glass baguette and allowing incubation with other petri dishes,
transferring fungal hyphae (fungal isolates) observed to develop out of the internal tissues during the incubation phase to petri dishes containing fresh medium for purification,
transferring fungal hyphae to fresh medium and coding of the axenic cultures which are pure cultures/fungal isolates obtained as a result of ensuring that the axenic cultures are purified by repeated sub culturing, based on an isolated host plant species and the internal tissues and routinely inoculated into petri dishes containing PDA (Potato Dextrose Agar) medium to ensure the continuity of cultures,
preparing stock cultures by inoculating the PDA medium to maintain the isolates,
incubating the stock cultures for 15 days at 23° C. in petri dishes containing YM 6.3 (Yeast-Malt Medium, pH 6.3) medium for identification studies of fungal isolates,
incubating the fungal isolates in petri dishes containing PDA for 5 days at 25° C. for biotransformation studies,
inoculating the fungi as a suspension culture into a biotransformation medium after incubation,
after inoculating, dissolving the resultant substrate of CA (Cycloastragenol), AG (astragenol) and CCG (Cycloanthogenol)) in DMSO (Dimethyl sulfoxide), adding to the medium and maintaining the substrate in submerged culture conditions,
biotransforming the substrate with the fungal isolate in a biotransformation medium,
removing resultant cells from resulting production broth under vacuum after incubation, then extracting the resulting filtrate with a volume of EtOAc equal to the volume of the broth,
combining resultant EtOAc phases, treating with anhydrous Na₂SO₄ and subsequent evaporating on a rotary evaporator at 40° C.,
obtaining telomerase activators which are new/novel molecules as final product.

2. The method for producing a telomerase activator according to claim 1, wherein a medium containing a rich/low nutrient containing antibiotic, PDA, MEA (Malt Extract Agar), RBC (Rose Bengal Chloramphenicol) agar and WA (Water Agar) is used in a fungus isolation to increase the endophyte isolation efficiency in the step of “cutting of internal tissues”.

3. The method for producing a telomerase activator according to claim 1, wherein the biotransformation medium is a broth containing 2% D (+) glucose, 0.5% yeast extract, 0.5% NaCl, 0.5% K₂HPO₄ (w/v) or Potato Dextrose Broth (PDB).

4. The method of producing a telomerase activator according to claim 1, wherein the plant comprises at least one of root, stem, leaf and flower of Astragalus condensatus and Astragalus angustifolius plants.

5. The method of producing a telomerase activator according to claim 1, wherein the substrate is dissolved in DMSO and added to the medium and biotransformation studies are carried out at 25° C. and 180 rpm at submerged culture conditions in the step of “dissolving the substrate”.

6. The method of producing a telomerase activator according to claim 1, wherein biotransformation of the substrate with the fungal isolate takes 10 days at a shaking speed of 180 rpm at 25° C. in a biotransformation medium in the step of “biotransforming the substrate”.

7. The method of producing a telomerase activator according to claim 1, wherein biotransformation of CA with at least one of the fungal isolates identified as Alternaria eureka, Neosartorya hiratsukae and Camarosporium laburnicola is performed in the step of “biotransforming the substrate”.

8. The method of producing a telomerase activator according to claim 1, wherein biotransformation of AG with at least one of the fungal isolates identified as Alternaria eureka and Camarosporium laburnicola is performed in the step of “biotransforming the substrate”.

9. The method of producing a telomerase activator according to claim 1, wherein biotransformation of CCG with the fungal isolate identified as Alternaria eureka is performed in the step of “biotransforming the substrate”.

10. Telomerase activators comprising molecules obtained by the method of claim 1 that effectively increase telomerase activity when administered to cells or tissues and given in at least one of the formulas of 1, 3, 4, 6, 10, 11, 12, 14, 16, 17, 19, 20, and 21;

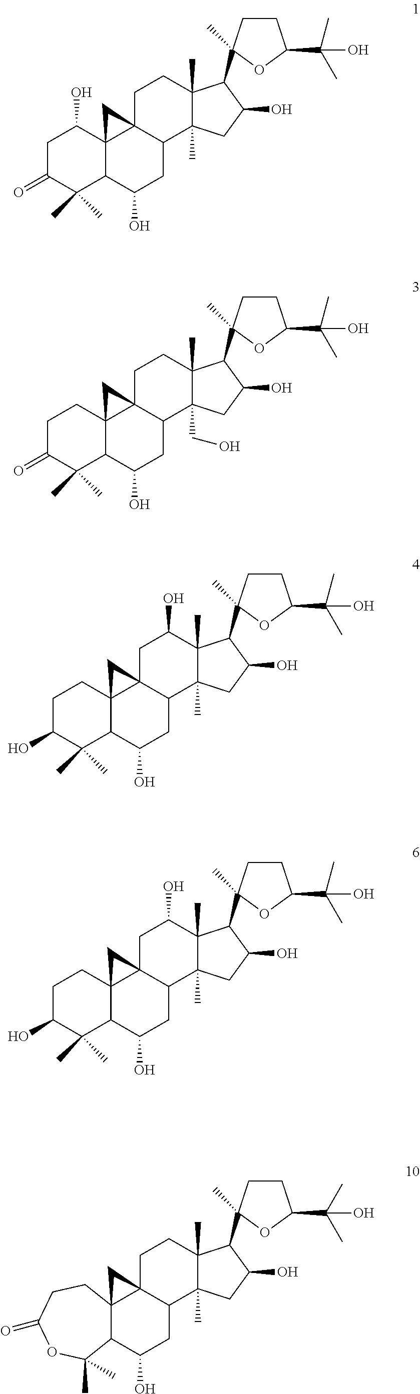

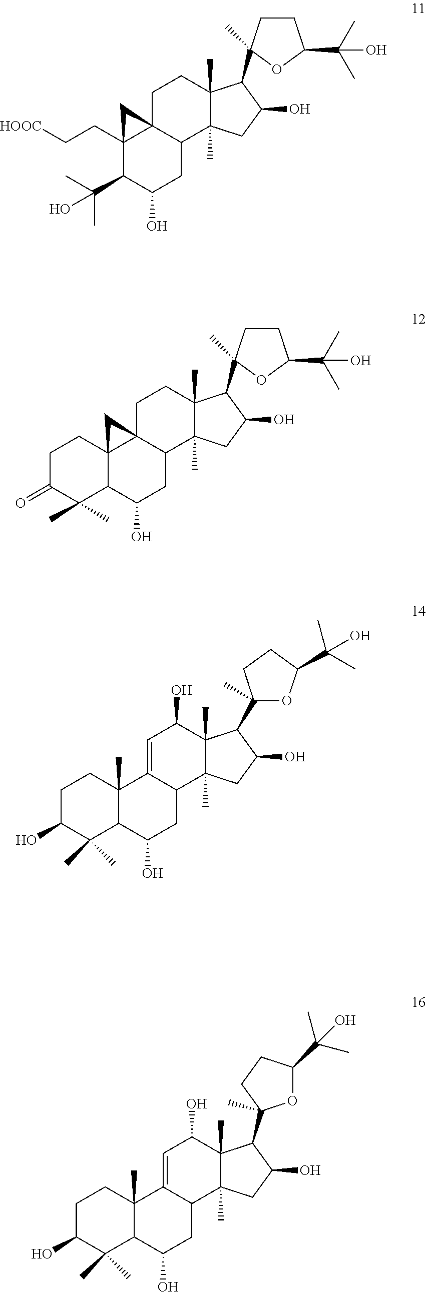

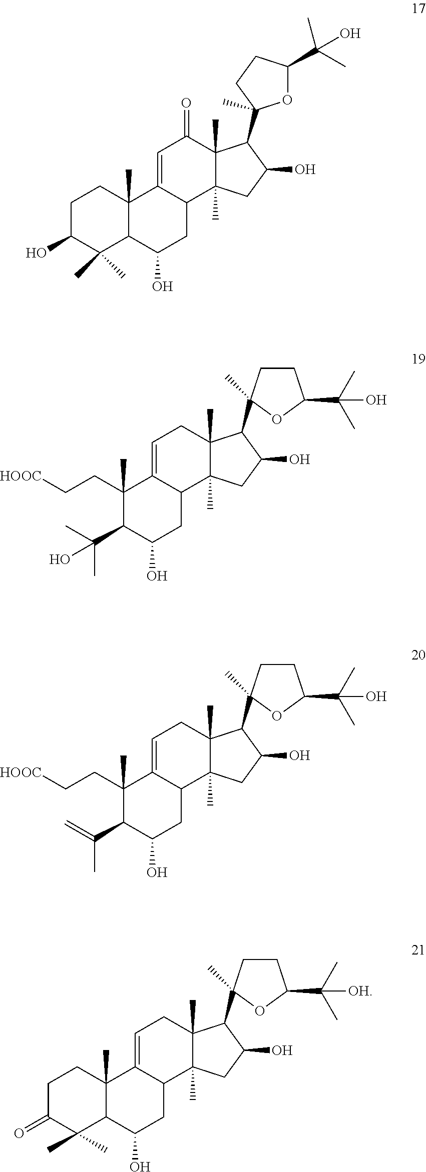

11. The telomerase activators of claim 10, wherein the telomerase activators comprise a pharmaceutically acceptable salt of at least one molecule disclosed in at least one formula of formula 1, 3, 4, 6, 10, 11, 12, 14, 16, 17, 19, 20, and 21 and which effectively increases telomerase activity when administered to cells or tissues.

12. The telomerase activators of claim 11, wherein a is telomerase activator is used to prevent/treat a condition or disease in mammalian cells or tissues that require increased telomerase activation.

13. The telomerase activators of claim 11, formulas (1, 3, 4, 6, 10, 11, 12, 14, 16, 17, 19, 20, 21) are given molecules and their salts, wherein a is telomerase activator is evaluated during in vitro production of stem cells or biological drugs (protein, antibody, etc.) which are used for regenerative or therapeutic purposes.

14. A telomerase activator produced by the method of claim 1, wherein the telomerase activator effectively increases telomerase activity when administered to cells or tissues and which comprises at least one molecule given in at least one of formulas 29-59 below; 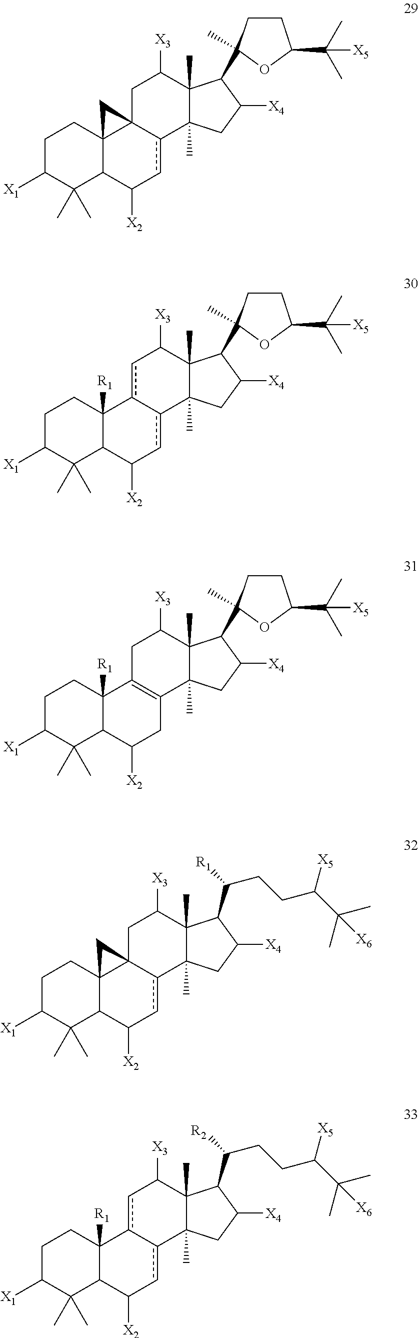 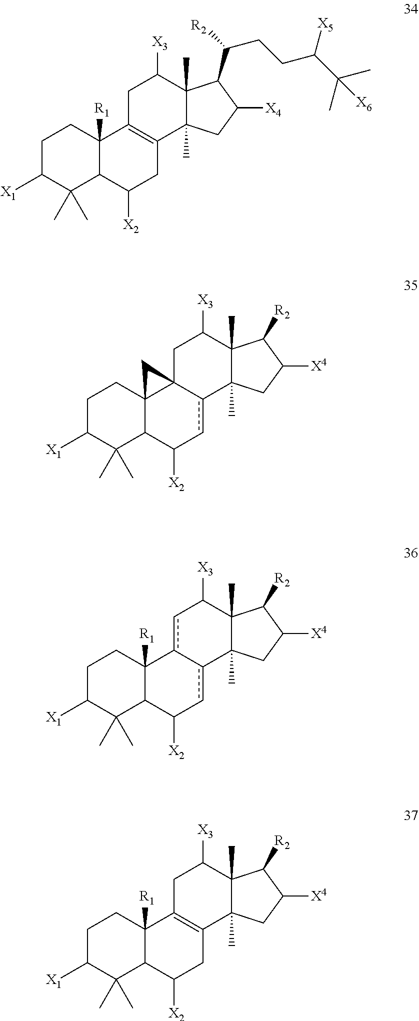 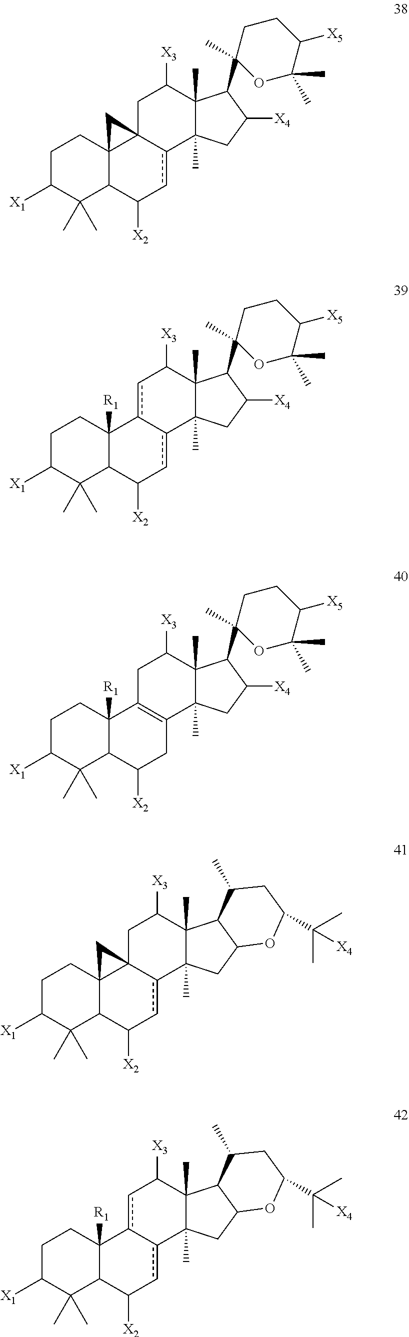 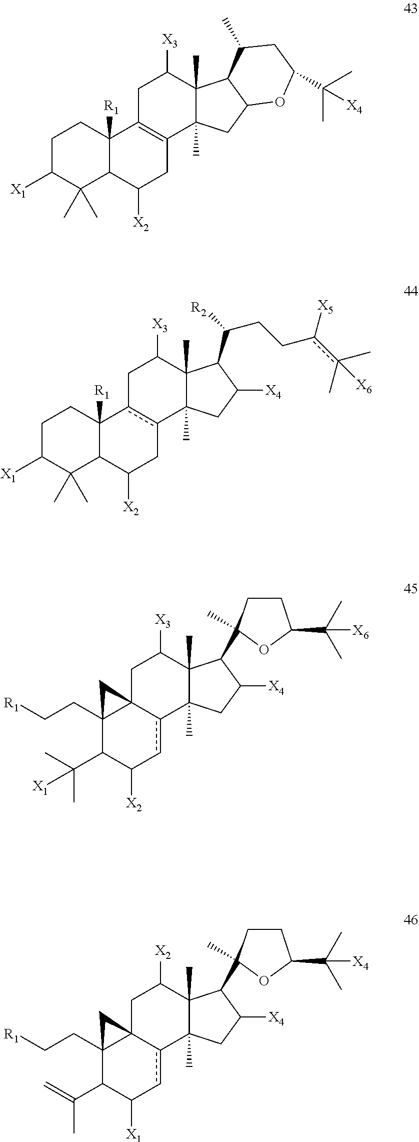 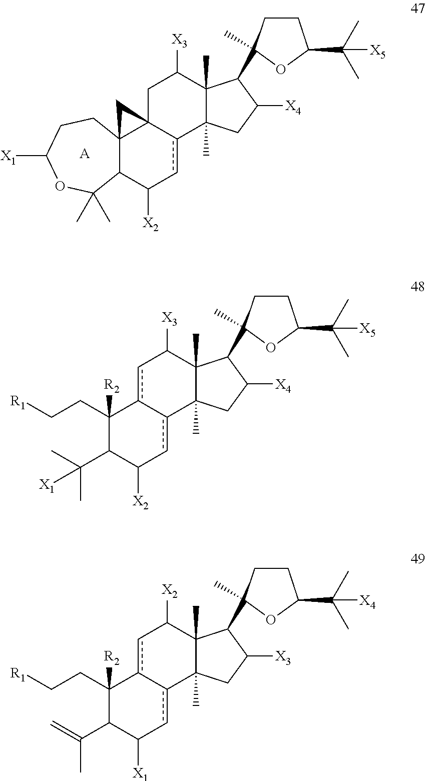 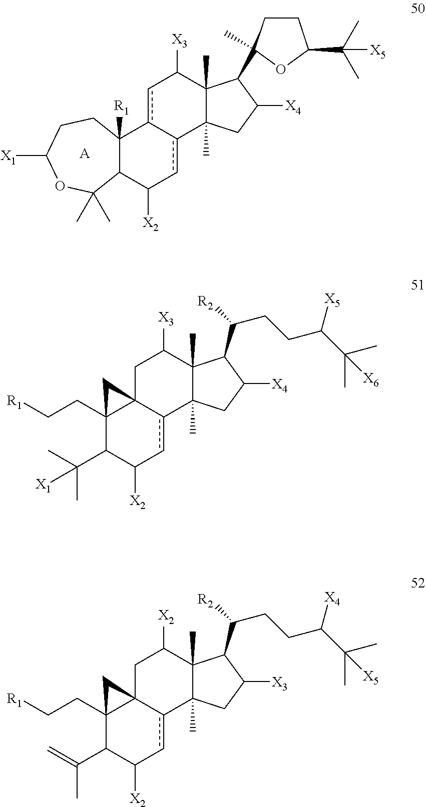 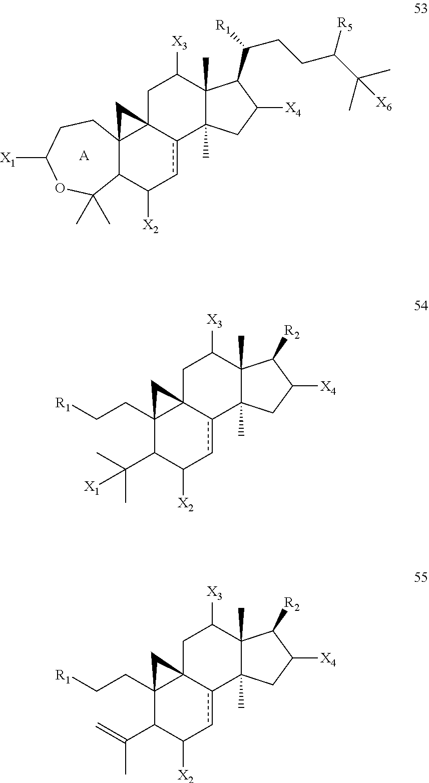 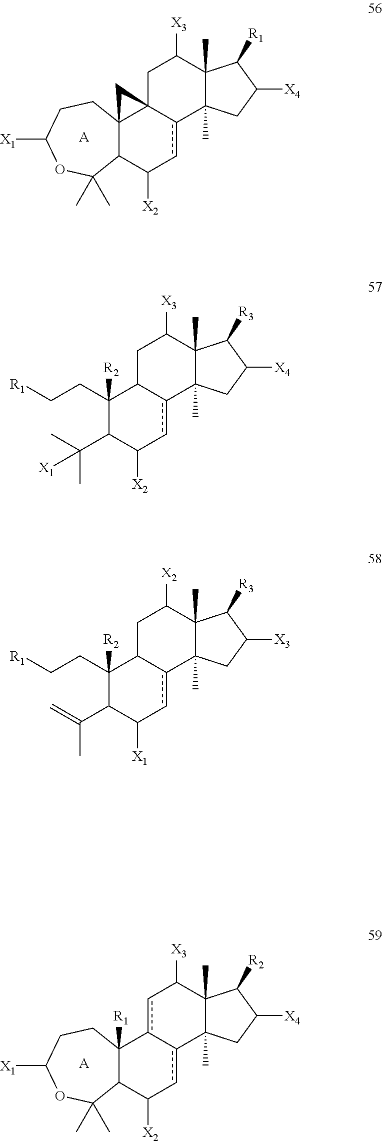
wherein each X₁, X₂, X₄, X₅ and X₆ are independently selected from hydrogen, hydroxy, alkoxy containing 1-6 carbons, acyloxy containing 1-6 carbons, keto and glycosides, wherein X₃ is independently selected from hydroxy, alkoxy containing 1-6 carbons, acyloxy containing 1-6 carbons, keto and glycoside, and
wherein each X₁, X₂, X₃, X₄, X₅ and X₆ independently have an alpha and beta configuration,
wherein if glycosylation is present on hydroxy groups, glycosylation on the sugar unit directly attaches to the main skeleton and the number of sugars on the glycosidic chain extends to a total of 3, and
wherein the groups R₁, R₂ and R₃ are independently selected from methyl and alcohol, aldehyde and carboxylic acid derivatives of methyl having different oxidation levels, and
wherein R₁, R₂ and R₃ are independently selected from alkoxy containing 1-6 carbons, acyloxy containing 1-6 carbons and glycoside on the hydroxy group present if the groups R₁, R₂ and R₃ are in the form of primary alcohols, and
wherein R₁, R₂ and R₃ are independently selected from ester or amide form with 1-16 carbon-bearing alcohols or amines if the groups R₁, R₂ and R₃ are in the form of carboxylic acid, and
if glycosidation is present on the primary alcohol present on the R₁, R₂ and R₃ groups, glycosylation on the sugar unit directly attaches to the main skeleton and the number of sugars on the glycosidic chain extends to a total of 3, wherein the R₁ group in structure 35 and the R₂ group in structures 36-37 are independently selected from hydrogen, hydroxy, alkyl chain containing from 2 to 6 carbons, haloalkyl chain containing 2-6 carbons, aryl, heteroaryl, monocyclic cycloalkyl chain containing from 3 to 8 carbons, bicyclic cycloalkyl chain containing from 4 to 8 carbons, the heterocyclic ring in the monocyclic structure containing 3-8 carbons, the heterocyclic ring in the bicyclic structure containing 4-8 carbons and wherein the chains thereof undergo substitution from 1 to 3 different points through the carbon atoms in the chain and the substitution comprises an alkyl substitution containing from 1 to 3 carbons, and
wherein there is at least one single or double bond between the ring carbons in the positions where the [symbol] symbol is present, and
wherein in the compounds 47, 50, 53, 56 and 59, the C-X₁ linkage extending from ring A comprises a double bond which occurs with at least one of the oxygen, nitrogen and sulfur atoms.

15. A method comprising the step of using the telomerase activators of claim 11 in the prevention or treatment of at least one condition or a disease present in a group of the diseases selected from the following or combinations thereof: viral infections, opportunistic infections, HIV, degenerative diseases, neurodegenerative diseases, degenerative diseases in bone, connective tissues and joints, diabetic retinopathy, macular degeneration, cardiovascular diseases, central and peripheral vascular diseases, Crohn's disease, immunological conditions, liver diseases, fibrosis, cirrhosis, lung diseases, pulmonary fibrosis, asthma, emphysema, chronic obstructive pulmonary diseases, hematopoietic disorders, anemia, thrombocytopenia, neutropenia, cytopenia, chronic inflammatory gastrointestinal diseases, Barret's esophagus, conditions associated with reduced proliferative capacity in stem cell or progenitor cells, bone marrow suppression diseases, aplastic anemia, myelodysplastic anemia, myelodysplastic syndrome, wounds, mucosal ulceration, keloid formation, hair loss, pigment loss, deep erosions and lesions, as well as severe acute and chronic discomforts, burns, abrasions, clefts and cuts, grafts, lesions, chronic venous ulcers, diabetic ulcers, cancer, genomic instability or increased mutations associated with telomerase or shortened telomer in pre-cancer cases, loss of tumor suppressor functions.
